(19) United States
(12) Patent Application Publication
(10) Pub. No.: US 2010/0212049 A1
(43) Pub. Date: Aug. 19, 2010

(54) Compositions and Methods of Use of Response Regulators

(75) Inventors: Norbert Brugière, Johnston, IA (US); Robert J. Meister, St. Peters, MO (US); Shoba Sivasankar, Urbandale, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International, Inc., Johnston, IA (US)

(21) Appl. No.: 12/700,143

(22) Filed: Feb. 4, 2010

Related U.S. Application Data
(63) Continuation of application No. 12/020,910, filed on Jan. 28, 2008, now abandoned.
Application No. 12/020,910 is a continuation of application No. 11/329,868, filed on Jan. 11, 2006, now abandoned.
(60) Provisional application No. 60/646,315, filed on Jan. 24, 2005.

(51) Int. Cl. A01H 3/00; C12N 15/82

(52) U.S. Cl. 800/290; 800/278; 435/468

(57) ABSTRACT

Methods and compositions for modulating plant development are provided. Methods employing type A Response Regulators (RR) are provided. The type A RR sequences are used in a variety of methods including modulating root development, modulating leaf and/or shoot development, modulating shoot regeneration from callus, modulating tolerance under abiotic stress, modulating yield, modulating cytokinin level/activity, and modulating plant responsiveness to cytokinin. Transformed plants, plant cell, tissues, seed, and expression vectors are also provided.

This application is a continuation of U.S. patent application Ser. No. 12/020,910, filed Jan. 28, 2008, pending, which is a continuation of Ser. No. 11/329,868, filed Jan. 11, 2006, pending, which claims priority to U.S. provisional patent application 60/646,315 filed Jan. 24, 2005, all of which are hereby incorporated by reference.

### FIELD OF THE INVENTION

The invention relates to the field of the genetic manipulation of plants, particularly the modulation of gene activity and development in plants.

### BACKGROUND OF THE INVENTION

Cytokinins are a class of N⁶ substituted purine derivative plant hormones that regulate cell division, as well as a large number of developmental events, such as plant growth, cell division, shoot initiation and development, root differentiation and development, leaf development, chloroplast development, and senescence (Mok, et al., (1995) Cytokinins Chemistry, Action and Function. CRC Press, Boca Raton, FLA, pp. 155-166).

Response Regulator (RR) proteins are part of the two-component signal transduction networks that are known to be involved in sensing cytokinin, ethylene and osmolarity in plant systems. A typical two-component system consists of a sensory histidine kinase and a response regulator. The first component, the sensory kinase, has an N-terminal input domain that detects changes in the external environment. This allows it to modulate intrinsic kinase or phosphatase activities at the C-terminal histidine kinase domain. The second component, the response regulator, has an N-terminal receiver domain with an invariant aspartate residue, and a C-terminal output domain. A ‘His-to-Asp phosphorelay’ between the two components allows the C-terminal output domain to initiate a downstream signaling cascade leading to environmental or hormonal adaptation. For a review, see, Haberer, et al., (2002) Plant Physiology 128:355-362; Takashi, et al., (2003) J. Plant Res. 116:221-231; and Hutchison, et al., (2002) The Plant Cell S57-S59.

In view of the influence of cytokinins on a wide variety of plant developmental processes, including root architecture, shoot and leaf development, and seed set, the ability to influence the responsiveness of a plant to cytokinin levels, and thereby drastically affect plant growth and productivity, is of great commercial value.

### BRIEF SUMMARY OF THE INVENTION

Compositions and methods of the invention employ Response Regulator (RR) polypeptides and polynucleotides that are involved in modulating plant development, morphology, and physiology. Compositions of the invention include a plant comprising a polynucleotide operably linked to a promoter that drives expression in the plant, wherein the polynucleotide comprises a nucleotide sequence selected from the group consisting of: (a) a nucleotide sequence comprising SEQ ID NO: 1, 3, 4, 6 or 11; (b) a nucleotide sequence encoding the amino acid sequence comprising SEQ ID NO: 2, 5 or 9; (c) a nucleotide sequence comprising at least 85% sequence identity to SEQ ID NO: 1, 4 or 11, wherein the polynucleotide encodes a polypeptide having response regulator activity; (d) a nucleotide sequence that hybridizes under stringent conditions to the complement of the nucleotide sequence of a), wherein the stringent conditions comprise hybridization in 50% formamide, 1 M NaCl, 1% SDS at 37° C., and a wash in 0.1×SSC at 60° C. to 65° C., wherein the polynucleotide encodes a polypeptide having response regulator activity; and (e) a nucleotide sequence comprising at least 50 consecutive nucleotides of SEQ ID NO: 1, 3, 4, 6, or 11 or a complement thereof.

In specific compositions, the promoter is a tissue-preferred promoter. In still further compositions, the tissue-preferred promoter is selected from the group consisting of an immature ear-preferred promoter, a kernel-preferred promoter, a seed-preferred promoter, a shoot-preferred promoter, a root-preferred promoter, and a leaf-preferred promoter.

In still other compositions, the plant has a modulated (decreased or increased) level and/or activity of the polypeptide selected from the group consisting of: (a) an amino acid sequence comprising SEQ ID NO: 2, 5 or 9; and (b) an amino acid sequence having at least 85% sequence identity to SEQ ID NO: 2, 5 or 9.

In other compositions, the plant has a modulation in plant yield, plant vigor, shoot growth, photosynthesis, leaf senescence, callus regeneration, stress tolerance, seed set, and/or root growth. In other compositions, the plant has a modulated responsiveness to a cytokinin.

Further compositions include an expression cassette comprising a polynucleotide operably linked to a promoter that drives expression in a plant. The polynucleotide comprises a nucleotide sequence selected from the group consisting of: (a) a nucleotide sequence comprising SEQ ID NO: 1, 3, 4 or 6; (b) a nucleotide sequence encoding the amino acid sequence comprising SEQ ID NO: 2, 5 or 9; (c) a nucleotide sequence comprising at least 85% sequence identity to SEQ ID NO: 1, 3, 4 or 6, wherein the polynucleotide encodes a polypeptide having response regulator activity; (d) a nucleotide sequence that hybridizes under stringent conditions to the complement of the nucleotide sequence of a), wherein the stringent conditions comprise hybridization in 50% formamide, 1 M NaCl, 1% SDS at 37° C., and a wash in 0.1×SSC at 60° C. to 65° C., wherein the polynucleotide encodes a polypeptide having response regulator activity; and, (e) a nucleotide sequence comprising at least 50 consecutive nucleotides of SEQ ID NO: 1, 3, 4 or 6, or a complement thereof.

Methods for modulating the level and/or activity of a polypeptide in a plant (a reduction or elimination of the level of the polypeptide or an increase in the level of the polypeptide) are provided and comprise introducing into the plant a polynucleotide comprising a nucleotide sequence selected from the group consisting of: (a) a nucleotide sequence comprising SEQ ID NO: 1, 4 or 11; (b) a nucleotide sequence encoding the amino acid sequence comprising SEQ ID NO: 2, 5 or 9; (c) a nucleotide sequence having at least 85% sequence identity to SEQ ID NO: 1, 4 or 11 wherein said polynucleotide encodes a polypeptide having response regulator activity; (d) a nucleotide sequence that hybridizes under stringent conditions to the complement of a polynucleotide of (a), wherein said stringent conditions comprise hybridization in 50% formamide, 1 M NaCl, 1% SDS at 37° C., and a wash in 0.1×SSC at 60° C. to 65° C.; and, (e) a nucleotide sequence comprising at least 50 consecutive nucleotides of SEQ ID NO: 1, 4 or 11 or a complement thereof.

In specific methods, the nucleotide sequence is operably linked to a tissue-specific promoter, a constitutive promoter, or an inducible promoter. In other methods, the tissue-preferred promoter is an immature ear-preferred promoter, a kernel-preferred promoter, a seed-preferred promoter, a leaf-preferred promoter, a root-preferred promoter, and a shoot-preferred promoter.

In other methods, modulation of the level and/or activity of the polypeptide modulates the stress tolerance, seed set during abiotic stress, plant yield, plant vigor, shoot growth, leaf senescence, shoot regeneration, and/or root growth.

### BRIEF DESCRIPTION OF FIGURES

FIG. 1 provides a time-course of Ckx1 induction by benzyladenine (BA) (left panel) and the relative abundance of Ckx1 and cyclophilin transcripts in maize leaf discs (right panel).

FIG. 2 provides an alignment of corn type A response regulators. Top to bottom in each set are ZmRR1 (AB031011, SEQ ID NO: 15), ZmRR2 (AB031012, SEQ ID NO: 16), ZmRR3 (AB042260, SEQ ID NO: 17), ZmRR4 (AB042261, SEQ ID NO: 18), ZmRR5 (AB042267, SEQ ID NO: 19), ZmRR6 (AB042268, SEQ ID NO: 20), ZmRR7 (AB042269, SEQ ID NO: 21), and Consensus (SEQ ID NO: 22). Conserved amino acids potentially participating in phosphorylation at the active site are boxed with a solid line (West and Stock (2001) Trends in Biochemical Sciences 26: 369-376) The putative phosphorylation site (Asp) is designated by an asterisk. A putative output domain present in ZmRR4, ZmRR5 and ZmRR6 is boxed in a dashed line. A putative nuclear localization signal is underlined.

FIG. 3 provides a PFAM alignment of ZmRR5 (SEQ ID NO: 2; AB042267) against the PFAM Response Regulator Domain (PF00072) (SEQ ID NO: 7).

FIG. 4 provides an alignment of ZmRR5 (SEQ ID NO: 2) against the SMART Response Regulator Domain (SM0048) (SEQ ID NO: 8).

FIG. 5 provides an alignment of ZmRR6 (SEQ ID NO: 5; AB042268) against the PFAM Response Regulator Domain (PF00072) (SEQ ID NO: 7).

FIG. 6 provides an alignment of ZmRR6 (SEQ ID NO: 5) against the SMART Response Regulator Domain (SM0048) (SEQ ID NO: 8).

FIG. 7 provides an alignment of ZmRR5 (SEQ ID NO: 2) and its insertional allele (SEQ ID NO: 9). The consensus sequence (SEQ ID NO: 23) is also shown. The native ZmRR5 sequence was aligned with the RR5 EST sequence p0128.cpicz20r which contains a 6-amino acid duplicated insertion within the output domain. Conserved amino acids potentially participating in phosphorylation at the active site as suggested by West and Stock (2001) Trends in Biochemical Sciences 26:369-376 are boxed in black, and the output domain is boxed in a dotted line. Amino acid duplications are indicated by asterisks.

FIG. 8 shows the weighted average of the fold-change of cytokinin-related genes in leaf samples of 8 separate transgenic events of PHP23835 carrying the ZM-RR5 transgene, relative to a bulk negative for the same transgene construct. Results shown for, left to right, ZmRR1, ZmRR2, putative cis-zog, ZmRR6, ZmRR5, cis-zog2, ZmCk×2, ZmRR4, ZmHK2, ZmRR7, ZmRR10, ZmCk×3, ZmIPT5. Out of 1624 sequences present on the Agilent® 8-pack chip, the greatest fold-change in the negative direction was obtained for ZmRR1.

FIG. 9 shows the fold-change of cytokinin-related genes in leaf samples of transgenic event number 8 of PHP23835 carrying the ZM-RR5 transgene, relative to a bulk negative for the construct. Left to right are ZmRR1, ZmRR2, ZmRR6, ZmRR5, ZmIPT5, ZmCk×2, ZmRR4, putative Ckx, ZmRR10, ZmRR8, ZmRR7, putative cis-zog, ZmCk×6, ZmHP2, ZmHK2.

### BRIEF DESCRIPTION OF THE SEQUENCES

The application provides details of response regulator sequences as shown in Table 1 below.

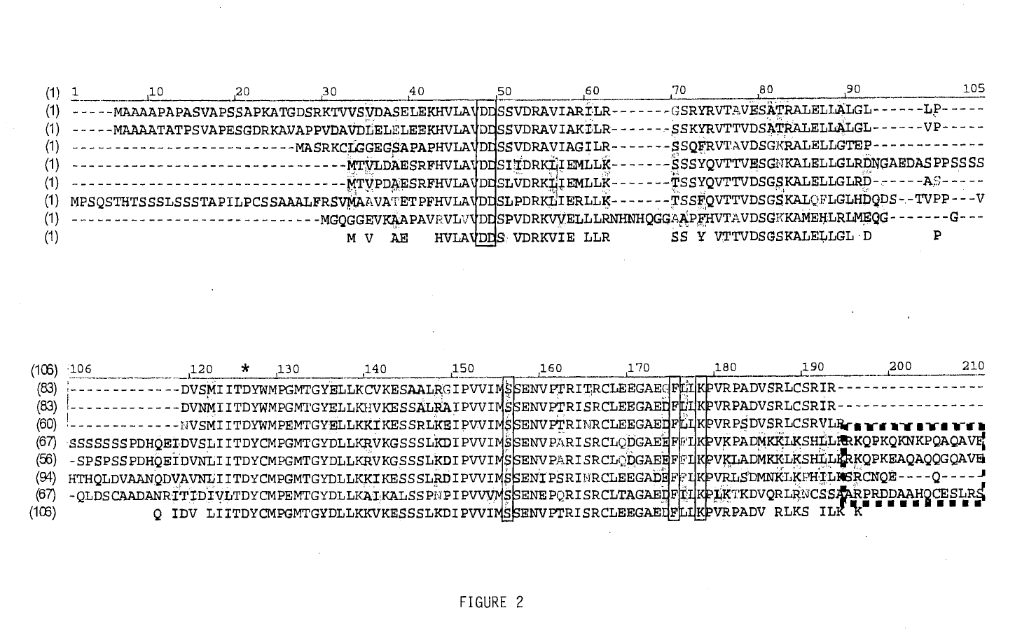

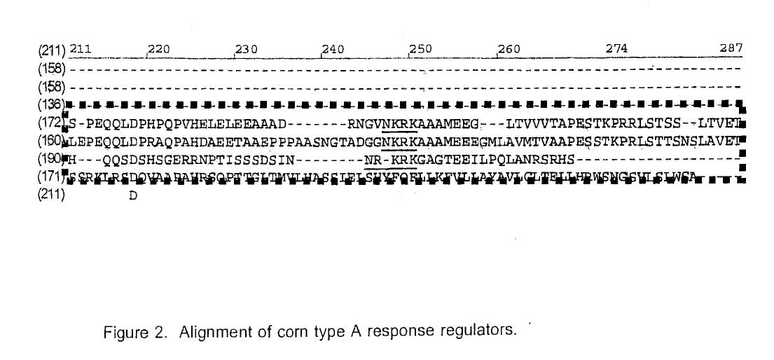

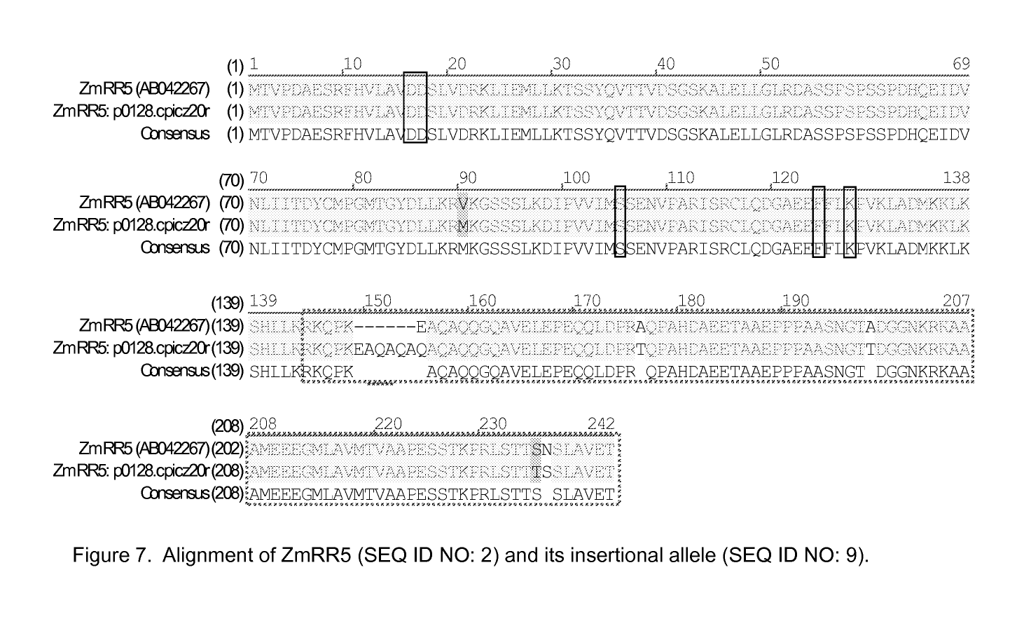

TABLE 1

| SEQ | Polynucleotide | | |
|---|---|---|---|
| ID | (pnt) or | | |
| NO: | polypeptide (ppt) | Length | Identification |
| 1 | pnt/ppt | 711/236 | ZmRR5 coding sequence (cds) |
| 2 | ppt | 236 | ZmRR5 polypeptide |
| 3 | pnt | 1654 | ZmRR5 full-length |
| 4 | pnt/ppt | 708/235 | ZmRR6 cds |
| 5 | ppt | 235 | ZmRR6 polypeptide |
| 6 | pnt | 1158 | ZmRR6 full-length |
| 7 | ppt | 125 | PFAM consensus for RR domain |
| 8 | ppt | 150 | SMART consensus for RR domain |
| 9 | ppt | 242 | ZmRR5 insertional allele |
| 10 | ppt | 232 | FIG. 7 consensus |
| 11 | pnt/ppt | 726/242 | cds of ZmRR5 insertional allele |
| 12 | pnt/ppt | 2061/686 | ZmRR10 cds of AB071695 |
| 13 | ppt | 236 | ZmRR5 (D75N) mutant |
| 14 | pnt | 1058 | ZmRR7 - AB042269 |
| 15 | ppt | 157 | ZmRR1 - AB031011 |
| 16 | ppt | 157 | ZmRR2 - AB031012 |
| 17 | ppt | 135 | ZmRR3 - AB042260 |
| 18 | ppt | 235 | ZmRR4 - AB042261 |
| 19 | ppt | 236 | ZmRR5 - AB042267 |
| 20 | ppt | 235 | ZmRR6 - AB042268 |
| 21 | ppt | 242 | ZmRR7 - AB042269 |
| 22 | ppt | 120 | Consensus from FIG. 2 |
| 23 | ppt | 232 | Consensus from FIG. 7 |

### DETAILED DESCRIPTION OF THE INVENTION

The present invention now will be described more fully hereinafter with reference to the accompanying examples, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the invention set forth herein will come to mind to one skilled in the art to which this invention pertains, having the benefit of the teachings presented in the descriptions and the drawings herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The articles “a” and “an” are used herein to refer to one or more than one (i.e., to at least one) of the grammatical object of the article. By way of example, “an element” means one or more than one element.

### OVERVIEW

Modulating shoot growth, root growth, stem tolerance, plant yield, and plant vigor can be achieved by targeting various individual genes, but the effect can be significantly enhanced by targeting an upstream step in a signal-transduction cascade specifically pertinent to growth and development. The two-component signal-transduction circuitry involved in cytokinin signaling is one such cascade, and the hierarchical position of the response regulators (RRs) in this pathway enhances their influence on plant responsiveness to cytokinin. Compositions and methods are provided to modulate plant development by influencing a RR of the cytokinin-signaling pathway.

### COMPOSITIONS

Compositions include plants having altered levels and/or activities of a type A response regulator (RR) polypeptide. In specific compositions, the plants have an altered level and/or activity of a type A RR polypeptide having the amino acid sequence set forth in SEQ ID NO: 2, 5 or 9 or an active variant or fragment thereof. Further provided are plants having an altered level and/or activity of the type A RR polypeptides encoded by a polynucleotide set forth in SEQ ID NOS: 1, 3, 4, 6 or 11 or an active variant or fragment thereof. The plants of invention can have a modulation in the stress tolerance of the plant, seed set during abiotic stress, plant yield, plant vigor, shoot growth, leaf senescence, shoot regeneration, and root growth. The RR sequences set forth in SEQ ID NO: 1-3 can be found in Genbank Accession Number AB042267 and the RR sequences set forth in SEQ ID NO: 4-6 can be found in Genbank Accession Number AB042268.

In specific embodiments, the plants of the invention have stably incorporated into their genomes a type A RR sequence. In further embodiments, the type A RR sequences are operably linked to a tissue-preferred promoter active in the plant. In other embodiments, plants genetically modified at a genomic locus encoding a type A RR polypeptide employed in the invention are provided. By “native genomic locus” is intended a naturally occurring genomic sequence. In some embodiments, the genomic locus is set forth in SEQ ID NO: 1 or 4. In still further embodiments, the genomic locus is modified to modulate the activity of the type A RR polypeptide. The term “genetically modified” as used herein refers to a plant or plant part that is modified in its genetic information by the introduction of one or more foreign polynucleotides, and that the insertion of the foreign polynucleotide leads to a phenotypic change in the plant. By “phenotypic change” is intended a measurable change in one or more cell functions. For example, plants having the genetic modification at the genomic locus encoding the type A RR polypeptide can show reduced or eliminated expression or activity of the type A RR polypeptide. Various methods to generate such a genetically modified genomic locus are described elsewhere herein, as are the variety of phenotypes that can result from the modulation of the level and/or activity of a type A RR sequence employed by the invention.

Modified plants are of interest, as are modified plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grain and the like. As used herein “grain” is intended the mature seed produced by commercial growers for purposes other than growing or reproducing the species. Progeny, variants, and mutants of the regenerated plants are also included within the scope of the invention, provided that these parts comprise the introduced nucleic acid sequences.

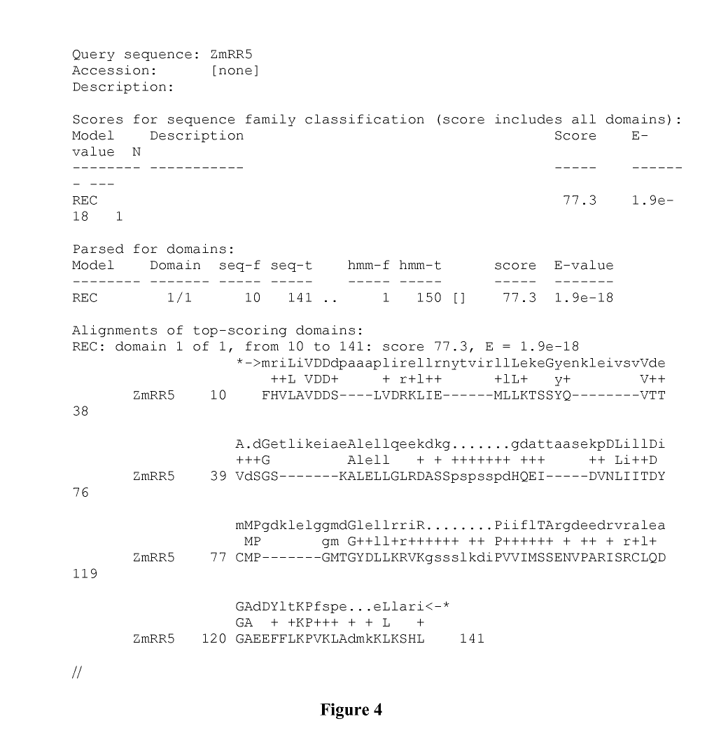

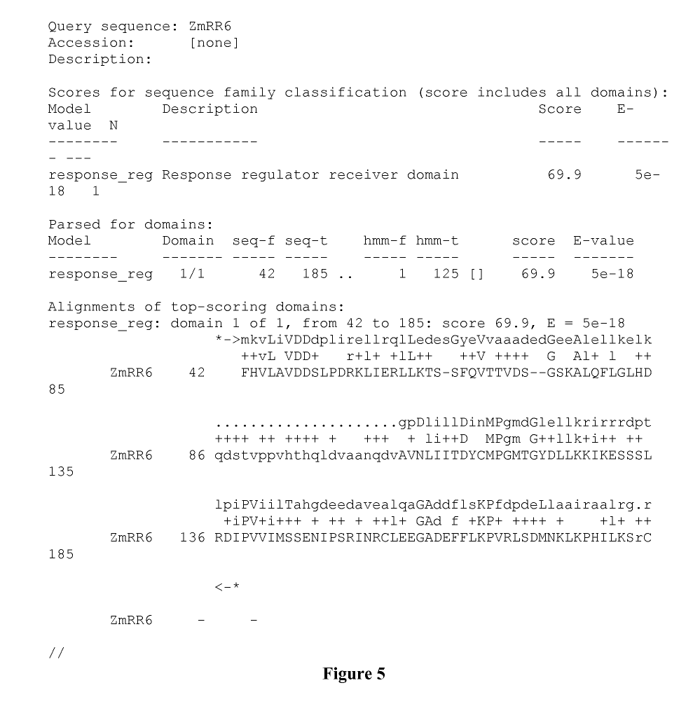

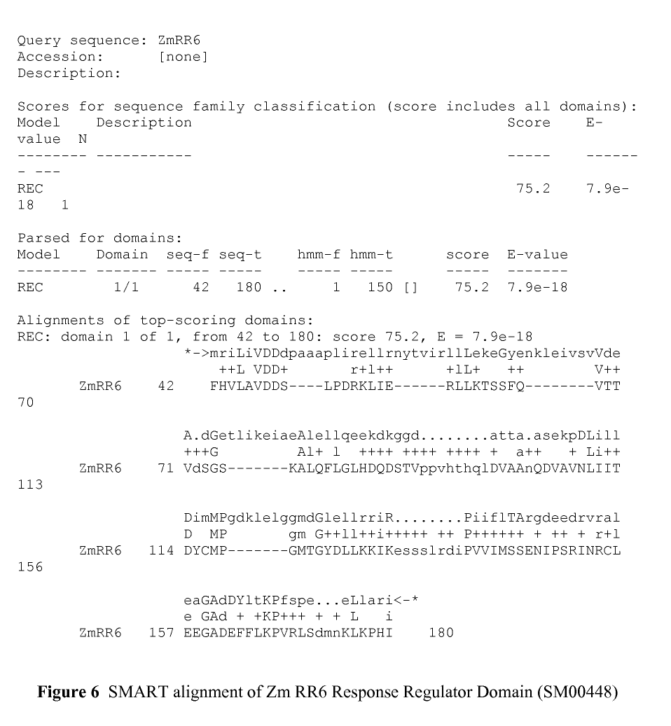

The type A RR polypeptides employed in the invention share sequence identity with members of the type A Response Regulator family of proteins. Changes in response regulator activity alter the responsiveness of the plant to cytokinins, and thus, response regulators influence many cytokinin-dependent processes. Members of the type-A class of response regulators (RRs) have been identified. See, for example, Schaller, et al., (2002) The Arabidopsis book. Eds. Somerville C R, Meyerowitz; Takashi, et al., (2003) J. Plant Res. 116:221-231, and Asakura, et al., (2003) Plant Molecular Biology 52:331-351, all of which are herein incorporated by reference. Type A RRs have a receiver domain with short N-terminal and C-terminal extensions. As shown in FIG. 2, conserved amino acids that potentially participate in phosphorylation at the active site are boxed with a solid line. The putative output domain is boxed in a dashed line, and the putative nuclear localization signal is underlined. The type A RR of the present invention further comprise domains having homology to the PFAM Response Regulatory Receiver Domain (PF00072), the SMART CheY-Homologous Receiver Domain (SM00448); and Prodom sp_Q9FRZ0 Maize_Q9FRZ0 domain (PD000039). Alignments of the ZmRR5 and the ZmRR6 polypeptides against the PFAM Response Regulator Domain (PF00072) and the SMART Response Regulator Domain (SM00448) are provided in FIGS. 4-6. See also, SEQ ID NOS: 7 and 8.

Type A RRs can have response regulatory activity. Response regulator activity includes, for example, an interaction with His-containing phosphotransfer proteins (HPs) which can be detected using assays such as the yeast two-hybrid system. See, for example, Asakura, et al., (2003) Plant Molecular Biology 52:331-351 and Clontech, Yeast Protocol Handbook. Response regulator activity also includes His-Asp phosphotransfer activity. His-Asp phosphotransfer activity occurs from a His-containing phosphotransfer protein (HP) to a RR. Such assays are known in the art. Briefly, His-tagged proteins expressed in E. coli are purified. Inner membrane vesicles of E. coli over-expressing ArcB can be used as the initial phosphor-donor (Tokishita, et al., (1990) J. Biochem 108:588-593; Sakakibara, et al., (1999) Plant Mol. Biol. 51:563-573; and, Suzuki, et al., (1998) Plant Cell Physiol. 105:1223-1229). On addition of the RR to the preparation of phosphor-HP, the phosphoryl group is transferred to the RR. See, for example, Asakura, et al., (2003) Plant Molecular Biology 52:331-351, herein incorporated by reference.

Response regulator activity also includes modulating the responsiveness of a plant to a cytokinin. By “modulating the responsiveness of a plant to a cytokinin” is intended any alteration in the development of the plant, when compared to a control plant, wherein the alterations arise due to either an enhanced sensitivity or a decreased sensitivity of the plant to cytokinin. As discussed in more detailed elsewhere herein, phenotypes associated with a modulated responsiveness to cytokinin include but are not limited to a modulation in root development, stress tolerance, shoot development, leaf development, leaf senescence, photosynthesis, callus regeneration, seed set, plant yield, or plant vigor.

Fragments and variants of the type A RR polynucleotides and proteins encoded thereby can be employed in the present invention. By “fragment” is intended a portion of the polynucleotide or a portion of the amino acid sequence and hence of the protein encoded thereby. Fragments of a polynucleotide may encode protein fragments that retain the biological activity of the native protein and hence retain response regulator activity. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides, up to the full-length polynucleotide encoding the proteins employed in the invention.

A fragment of a type A RR polynucleotide that encodes a biologically active portion of a type A RR protein employed in the invention will encode at least 15, 25, 30, 50, 75, 100, 125, 150, 175, 200, 220 or 225 contiguous amino acids, or up to the total number of amino acids present in a full-length type A RR protein of the invention (for example, 236 or 235 amino acids for SEQ ID NO: 2 and 5, respectively). Fragments of a type A RR polynucleotide that are useful as hybridization probes or PCR primers generally need not encode a biologically active portion of a type A RR protein.

Thus, a fragment of a type A RR polynucleotide may encode a biologically active portion of a type A RR protein, or it may be a fragment that can be used as a hybridization probe or PCR primer using methods disclosed below. A biologically active portion of a type A RR protein can be prepared by isolating a portion of one of the type A RR polynucleotide employed in the invention, expressing the encoded portion of the type A RR protein (e.g., by recombinant expression in vitro), and assessing the activity of the encoded portion of the type A RR protein. Polynucleotides that are fragments of a type A RR nucleotide sequence comprise at least 16, 20, 50, 75, 100, 150, 200, 250, 300, 350, 500, 550, 500, 550, 600, 650, 700, 800, 900, 1,000, 1,100 nucleotides, or up to the number of nucleotides present in a full-length type A RR polynucleotide disclosed herein (for example, 1158 and 1654 nucleotides for SEQ ID NOS: 6 and 3, respectively).

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the type A RR polypeptides of the invention. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a type A RR protein employed in the invention. Generally, variants of a particular polynucleotide of the invention will have at least about 50%, 55%, 50%, 55%, 60%, 65%, 70%, 75%, 80%, 85%, 90%, 91%, 92%, 93%, 95%, 95%, 96%, 97%, 98%, 99% or more sequence identity to that particular polynucleotide as determined by sequence alignment programs and parameters described elsewhere herein.

Variants of a particular polynucleotide employed in the invention (i.e., the reference polynucleotide) can also be evaluated by comparison of the sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 2, 5 or 9 is encompassed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 50%, 55%, 50%, 55%, 60%, 65%, 70%, 75%, 80%, 85%, 90%, 91%, 92%, 93%, 95%, 95%, 96%, 97%, 98%, 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, response regulator activity as described herein. Such variants may result from, for example, genetic polymorphism or from human manipulation. Biologically active variants of a native type A RR protein of the invention will have at least about 50%, 55%, 50%, 55%, 60%, 65%, 70%, 75%, 80%, 85%, 90%, 91%, 92%, 93%, 95%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 5, 3, 2 or even 1 amino acid residue. Variants of the invention include the amino acid sequence and nucleotide sequence set forth in SEQ ID NO: 9 and 11.

The proteins employed in the methods of the invention may be altered in various ways including amino acid substitutions, deletions, truncations, and insertions. Methods for such manipulations are generally known in the art. For example, amino acid sequence variants and fragments of the type A RR proteins can be prepared by mutations in the DNA. Methods for mutagenesis and polynucleotide alterations are well known in the art. See, for example, Kunkel (1985) Proc. Natl. Acad. Sci. USA 82:588-592; Kunkel, et al., (1987) Methods in Enzymol. 155:367-382; U.S. Pat. No. 5,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be optimal. Variants of type A RR polypeptides can also include the addition of domains found in type B response regulators.

Thus, the genes and polynucleotides employed in the invention include both the naturally occurring sequences as well as mutant forms. Likewise, the proteins employed in the invention encompass naturally occurring proteins as well as variations and modified forms thereof. Such variants will continue to possess the desired response regulator activity. Obviously, the mutations that will be made in the DNA encoding the variant must not place the sequence out of reading frame and optimally will not create complementary regions that could produce secondary mRNA structure. See, EP Patent Application Publication Number 75,555.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by an interaction with His-containing phosphotransfer proteins (HPs), His-Asp phosphotransfer activity, and/or a modulation in the responsiveness of a plant to a cytokinin. Assays for detecting such activity are described in detail elsewhere herein.

Fragments and variants of the type A RR polynucleotides and proteins encoded thereby can be employed in the present invention. By “fragment” is intended a portion of the polynucleotide or a portion of the amino acid sequence and hence protein encoded thereby. Fragments of a polynucleotide may encode protein fragments that retain the biological activity of the native protein and hence retains response regulator activity. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the proteins employed in the invention.

Variant polynucleotides and proteins also encompass sequences and proteins derived from a mutagenic and recombinogenic procedure such as DNA shuffling. With such a procedure, one or more different RR coding sequences can be manipulated to create a new type A RR possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the type A RR gene of the invention and other known RR genes to obtain a new gene coding for a protein with an improved property of interest, such as an increased Kₘ in the case of an enzyme. Strategies for such DNA shuffling are known in the art. See, for example, Stemmer (1995) Proc. Natl. Acad. Sci. USA 91:10757-10751; Stemmer (1995) Nature 370:389-391; Crameri, et al., (1997) Nature Biotech. 15:536-538; Moore, et al., (1997) J. Mol. Biol. 272:336-357; Zhang, et al., (1997) Proc. Natl. Acad. Sci. USA 95:5505-5509; Crameri, et al., (1998) Nature 391:288-291; and U.S. Pat. Nos. 5,605,793 and 5,837,558.

The polynucleotides employed in the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire type A RR sequences set forth in SEQ ID NO: 1, 4 or 11 or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 95%, 95%, 96%, 97%, 98%, 99%, or greater sequence identity. Functions of orthologs are often highly conserved among species. Thus, isolated polynucleotides that encode for a type A RR protein and which hybridize under stringent conditions to the sequence of SEQ ID NO: 1 or 4, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook, et al., (1989) Molecular Cloning: A Laboratory Manual (2d ed., Cold Spring Harbor Laboratory Press, Plainview, N.Y.). See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or an other detectable marker. Thus, for example, probes for hybridization can be made by labeling synthetic oligonucleotides based on the type A RR polynucleotides of the invention. Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, N.Y.).

For example, the entire type A RR polynucleotide disclosed herein, or one or more portions thereof, may be used as a probe capable of specifically hybridizing to corresponding type A RR polynucleotide and messenger RNAs. To achieve specific hybridization under a variety of conditions, such probes include sequences that are unique among type A RR polynucleotide sequences and are optimally at least about 10 nucleotides in length, and most optimally at least about 20 nucleotides in length. Such probes may be used to amplify corresponding type A RR polynucleotide from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook, et al., (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 50 to 55% formamide, 1.0 M NaCl, 1%) SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 25 hours, usually about 5 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1985) Anal. Biochem. 138:267-285: Tₘ=81.5° C.+16.6 (log M)+0.51 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 5° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 15, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 55° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel, et al., eds. (1995) Current Protocols in Molecular Biology, Chapter 2 (Greene Publishing and Wiley-Interscience, New York). See, Sambrook, et al., (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 50, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 5:11-17; the local alignment algorithm of Smith, et al., (1981) Adv. Appl. Math. 2:582; the global alignment algorithm of Needleman and Wunsch (1970) J. Mol. Biol. 58:553-553; the search-for-local alignment method of Pearson and Lipman (1988) Proc. Natl. Acad. Sci. 85:2555-2558; the algorithm of Karlin and Altschul (1990) Proc. Natl. Acad. Sci. USA 872265, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-255 (1988); Higgins, et al., (1989) CABIOS 5:151-153; Corpet, et al., (1988) Nucleic Acids Res. 16:10881-90; Huang, et al., (1992) CABIOS 8:155-65; and Pearson, et al., (1995) Meth. Mol. Biol. 25:307-331. The ALIGN program is based on the algorithm of Myers and Miller (1988) supra. A PAM120 weight residue table, a gap length penalty of 12, and a gap penalty of 5 can be used with the ALIGN program when comparing amino acid sequences. The BLAST programs of Altschul, et al., (1990) J. Mol. Biol. 215:503 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, www.ncbi.nlm.nih.gov. Alignment may also be performed manually by inspection.

Unless otherwise stated, sequence identity/similarity values provided herein refer to the value obtained using GAP Version 10 using the following parameters: identity and % similarity for a nucleotide sequence using GAP Weight of 50 and Length Weight of 3, and the nwsgapdna.cmp scoring matrix; % identity and similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 58:553-553, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 5, 5, 6, 7, 8, 9, 10, 15, 20, 25, 30, 35, 50, 55, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 5 kb, 3 kb, 2 kb, 1 kb, 0.5 kb or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

### Methods

### I. Providing Sequences

The sequences of the present invention can be introduced/expressed in a host cell such as bacteria, yeast, insect, mammalian, or optimally plant cells. It is expected that those of skill in the art are knowledgeable in the numerous systems available for the introduction of a polypeptide or a nucleotide sequence of the present invention into a host cell. No attempt to describe in detail the various methods known for providing proteins in prokaryotes or eukaryotes will be made.

By “host cell” is meant a cell, which comprises a heterologous nucleic acid sequence of the invention. Host cells may be prokaryotic cells such as E. coli, or eukaryotic cells such as yeast, insect, amphibian, or mammalian cells. Host cells can also be monocotyledonous or dicotyledonous plant cells. In one embodiment, the monocotyledonous host cell is a maize host cell.

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The type A RR polynucleotide employed in the invention can be provided in expression cassettes for expression in the plant of interest. The cassette will include 5′ and 3′ regulatory sequences operably linked to a type A RR polynucleotide. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the type A RR polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a type A RR polynucleotide of the invention, and a transcriptional and translational termination region (i.e., termination region) functional in plants. The regulatory regions (i.e., promoters, transcriptional regulatory regions, and translational termination regions) and/or the type A RR polynucleotide of the invention may be native/analogous to the host cell or to each other. Alternatively, the regulatory regions and/or the type A RR polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change the expression levels of the type A RR in the plant or plant cell. Thus, the phenotype of the plant or plant cell can be altered.

The termination region may be native with the transcriptional initiation region, may be native with the operably linked type A RR polynucleotide of interest, may be native with the plant host, or may be derived from another source (i.e., foreign or heterologous) to the promoter, the type A RR polynucleotide of interest, the plant host, or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:151-155; Proudfoot, (1991) Cell 65:671-675; Sanfacon, et al., (1991) Genes Dev. 5:151-159; Mogen, et al. (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,536,391, and Murray, et al., (1989) Nucleic Acids Res. 17:577-598, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 155:9-20), and human immunoglobulin heavy-chain binding protein (BiP) (Macejak, et al., (1991) Nature 353:90-95); untranslated leader from the coat protein mRNA of alfalfa mosaic virus (AMV RNA 5) (Jobling, et al., (1987) Nature 325:622-625); tobacco mosaic virus leader (TMV) (Gallie, et al., (1989) in Molecular Biology of RNA, ed. Cech (Liss, New York), pp. 237-256); and maize chlorotic mottle virus leader (MCMV) (Lommel, et al., (1991) Virology 81:382-385). See also, Della-Cioppa, et al., (1987) Plant Physiol. 85:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,5-dichlorophenoxyacetate (2,5-D). Additional selectable markers include phenotypic markers such as β-galactosidase and fluorescent proteins such as green fluorescent protein (GFP) (Su, et al., (2005) Biotechnol Bioeng 85:610-9 and Fetter, et al., (2005) Plant Cell 16:215-28), cyan florescent protein (CYP) (Bolte, et al., (2005) J. Cell Science 117:953-55 and Kato, et al., (2002) Plant Physiol 129:913-52), and yellow florescent protein (PhiYFP™ from Evrogen, see, Bolte, et al., (2005) J. Cell Science 117:953-55). For additional selectable markers, see generally, Yarranton, (1992) Curr. Opin. Biotech. 3:506-511; Christopherson, et al., (1992) Proc. Natl. Acad. Sci. USA 89:6315-6318; Yao, et al., (1992) Cell 71:63-72; Reznikoff, (1992) Mol. Microbiol. 6:2519-2522; Barkley, et al., (1980) in The Operon, pp. 177-220; Hu, et al., (1987) Cell 58:555-566; Brown, et al., (1987) Cell 59:603-612; Figge, et al., (1988) Cell 52:713-722; Deuschle, et al., (1989) Proc. Natl. Acad. Sci. USA 86:5500-5505; Fuerst, et al., (1989) Proc. Natl. Acad. Sci. USA 86:2559-2553; Deuschle, et al., (1990) Science 258:580-583; Gossen, (1993) Ph.D. Thesis, University of Heidelberg; Reine, et al., (1993) Proc. Natl. Acad. Sci. USA 90:1917-1921; Labow, et al., (1990) Mol. Cell. Biol. 10:3353-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:5657-5653; Hillenand-Wissman, (1989) Topics Mol. Struc. Biol. 10:153-162; Degenkolb, et al., (1991) Antimicrob. Agents Chemother. 35:1591-1595; Kleinschnidt, et al., (1988) Biochemistry 27:1095-1105; Bonin, (1993) Ph.D. Thesis, University of Heidelberg; Gossen, et al., (1992) Proc. Natl. Acad. Sci. USA 89:5557-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 335:721-725. Such disclosures are herein incorporated by reference. The above list of selectable marker genes is not meant to be limiting. Any selectable marker gene can be used in the present invention.

A number of promoters can be used in the practice of the invention, including the native promoter of the polynucleotide sequence of interest. The promoters can be selected based on the desired outcome. The nucleic acids can be combined with constitutive, tissue-preferred, inducible, or other promoters for expression in plants.

Such constitutive promoters include, for example, the core promoter of the Rsyn7 promoter and other constitutive promoters disclosed in WO 99/53838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1985) EMBO J. 3:2723-2730); ALS promoter (U.S. Pat. No. 5,659,026), and the like. Other constitutive promoters include, for example, those disclosed in U.S. Pat. Nos. 5,608,159; 5,608,155; 5,605,121; 5,569,597; 5,566,785; 5,399,680; 5,268,563; 5,608,152 and 6,177,611.

Tissue-preferred promoters can be utilized to target enhanced type A RR expression within a particular plant tissue. By “tissue-preferred” is intended to mean that expression is predominately in a particular tissue, albeit not necessarily exclusively in that tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen. Genet. 255(3):337-353; Russell, et al., (1997) Transgenic Res. 6(2):157-168; Rinehart, et al., (1996) Plant Physiol. 112(3):1331-1351; Van Camp, et al., (1996) Plant Physiol. 112(2):525-535; Canevascini, et al., (1996) Plant Physiol. 112(2):513-525; Yamamoto, et al., (1995) Plant Cell Physiol. 35(5):773-778; Lam, (1995) Results Probl. Cell Differ. 20:181-196; Orozco, et al., (1993) Plant Mol. Biol. 23(6):1129-1138; Matsuoka, et al., (1993) Proc Natl. Acad. Sci. USA 90(20):9586-9590; and Guevara-Garcia, et al., (1993) Plant J. 5(3):595-505. Such promoters can be modified, if necessary, for weak expression. See, also, US Patent Application Number 2003/0074698, herein incorporated by reference.

Leaf-preferred promoters are known in the art. See, for example, Yamamoto, et al., (1997) Plant J. 12(2):255-265; Kwon, et al., (1995) Plant Physiol. 105:357-67; Yamamoto, et al., (1995) Plant Cell Physiol. 35(5):773-778; Gotor, et al., (1993) Plant J. 3:509-18; Orozco, et al., (1993) Plant Mol. Biol. 23(6):1129-1138; Baszczynski, et al., (1988) Nucl. Acid Res. 16:5732; Mitra, et al., (1995) Plant Molecular Biology 26:35-93; Kayaya, et al., (1995) Molecular and General Genetics 258:668-675; and Matsuoka, et al., (1993) Proc. Natl. Acad. Sci. USA 90(20):9586-9590. Senescence regulated promoters are also of use, such as, SAM22 (Crowell, et al., (1992) Plant Mol. Biol. 18:559-566). See also, U.S. Pat. No. 5,589,052, herein incorporated by reference.

Shoot-preferred promoters include, shoot meristem-preferred promoters such as promoters disclosed in Weigal, et al., (1992) Cell 69:853-859; Accession Number AJ131822; Accession Number Z71981; Accession Number AF059870, the ZAP promoter (U.S. patent application Ser. No. 10/387,937), the maize tbl promoter (Wang, et al., (1999) Nature 398:236-239, and shoot-preferred promoters disclosed in McAvoy, et al., (2003) Acta Hort. (ISHS) 625:379-385.

Root-preferred promoters are known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger, et al., (1990) Plant Mol. Biol. 15(3):533-553 (root-specific promoter of the mannopine synthase (MAS) gene of Agrobacterium tumefaciens); and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-651, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see, Plant Science (Limerick) 79(1):69-76). They concluded that enhancer and tissue-preferred DNA determinants are dissociated in those promoters. Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):353-350). The TR1′ gene, fused to nptII (neomycin phosphotransferase II) showed similar characteristics. Additional root-preferred promoters include the VfENOD-GRP3 gene promoter (Kuster, et al., (1995) Plant Mol. Biol. 29(5):759-772); roIB promoter (Capana, et al., (1995) Plant Mol. Biol. 25(5):681-691; and the CRWAQ81 root-preferred promoter with the ADH first intron (US Provisional Application Number 60/509,878, filed Oct. 9, 2003, herein incorporated by reference). See also, U.S. Pat. Nos. 5,837,876; 5,750,386; 5,633,363; 5,559,252; 5,501,836; 5,110,732 and 5,023,179.

“Seed-preferred” promoters include “seed-specific” promoters (those promoters active during seed development (i.e., kernel-preferred promoters) such as promoters of seed storage proteins). Seed-specific promoters include those that are active either before or after pollination, or those that are active independent of pollination. Seed-preferred promoter also include “seed-germinating” promoters (those promoters active during seed germination). See, Thompson, et al., (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see, WO 00/11177 and U.S. Pat. No. 6,225,529; herein incorporated by reference); PCNA2 (U.S. patent application Ser. No. 10/388,359, filed Mar. 13, 2003) and, CKX1-2 (US Patent Application Publication Number 20020152500). Gamma-zein is an endosperm-specific promoter. Globulin-1 (Glob-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also, WO 00/12733, where seed-preferred promoters from end1 and end2 genes are disclosed and WO 01/21783 and U.S. Pat. No. 6,403,862, where the Zm40 promoter is disclosed; both herein incorporated by reference. Embryo-specific promoters include ESR (US Patent Application Publication Number 20040210960) and lecl (U.S. patent application Ser. No. 09/718,754, filed Nov. 22, 2000). Additional embryo specific promoters are disclosed in Sato, et al., (1996) Proc. Natl. Acad. Sci. 93:8117-8122; Nakase, et al., (1997) Plant J 12:235-56; and Postma-Haarsma, et al., (1999) Plant Mol. Biol. 39:257-71. Endosperm-preferred promoters include eppl and eep2 as disclosed in US Patent Application Publication Number 20040237147. Additional endosperm specific promoters are disclosed in Albani, et al., (1985) EMBO 3:1505-15; Albani, et al., (1999) Theor. Appl. Gen. 98:1253-62; Albani, et al., (1993) Plant J. 5:353-55; Mena, et al., (1998) The Plant Journal 116:53-62, and Wu, et al., (1998) Plant Cell Physiology 39:885-889. Immature ear tissue-preferred promoters can also be employed.

Dividing cell or meristematic tissue-preferred promoters have been disclosed in Ito, et al., (1995) Plant Mol. Biol. 25:863-878; Reyad, et al., (1995) Mo. Gen. Genet. 258:703-711; Shaul, et al., (1996) Proc. Natl. Acad. Sci. 93:5868-5872; Ito, et al., (1997) Plant J. 11:983-992; Treh in, et al., (1997) Plant Molecular Biology 35:667-672; Zag1 (Schmidt, et al., (1993) The Plant Cell 5:729-37) and Zag2 from maize (Theissen, et al., (1995) Gene 156:155-166) GenBank Accession Number X80206; and Hubbard, et al., (2002) Genetics 162:1927-1935, all of which are herein incorporated by reference.

Inflorescence-preferred promoters include the promoter of chalcone synthase (Van der Meer, et al., (1990) Plant Mol. Biol. 15:95-109), LAT52 (Twell, et al., (1989) Mol. Gen. Genet. 217:250-255), pollen specific genes (Albani, et al., (1990) Plant Mol. Biol. 15:605, Zm13 (Buerrero, et al., (1993) Mol. Gen. Genet. 225:161-168), maize pollen-specific gene (Hamilton, et al., (1992) Plant Mol. Biol. 18:211-218), sunflower pollen expressed gene (Baltz, et al., (1992) The Plant Journal 2:713-721), B. napus pollen specific genes (Arnoldo, et al., (1992) J. Cell. Biochem, Abstract Number Y101205). Immature ear tissue-preferred promoters can also be employed.

Stress inducible promoters include salt/water stress-inducible promoters such as P5CS (Zang, et al., (1997) Plant Sciences 129:81-89); cold-inducible promoters, such as, cor15a (Hajela, et al., (1990) Plant Physiol. 93:1256-1252), cor15b (Wilhelm, et al., (1993) Plant Mol Biol 23:1073-1077), wsc120 (Ouellet, et al., (1998) FEBS Lett. 523:325-328), ci7 (Kirch, et al., (1997) Plant Mol. Biol. 33:897-909), ci21A (Schneider, et al., (1997) Plant Physiol. 113:335-55); and MLIP15 (U.S. Pat. No. 6,479,734) drought-inducible promoters, such as, Trg-31 (Chaudhary, et al., (1996) Plant Mol. Biol. 30:1257-57), rd29 (Kasuga, et al., (1999) Nature Biotechnology 18:287-291); osmotic inducible promoters, such as, Rab17 (Vilardell, et al., (1991) Plant Mol. Biol. 17:985-93) and osmotin (Raghothama, et al., (1993) Plant Mol Biol 23:1117-28); and, heat inducible promoters, such as, heat shock proteins (Barros, et al., (1992) Plant Mol. 19:665-75; Marrs, et al., (1993) Dev. Genet. 15:27-51), senescence inducible promoters, such as SEE1 (GB_AJ494982), and smHSP (Waters, et al., (1996) J. Experimental Botany 57:325-338). Other stress-inducible promoters include rip2 (U.S. Pat. No. 5,332,808 and US Publication Number 2003/0217393) and rp29a (Yamaguchi-Shinozaki, et al., (1993) Mol. Gen. Genetics 236:331-350).

Nitrogen-responsive promoters can also be used in the methods of the invention. Such promoters include, but are not limited to, the 22 kDa Zein promoter (Spena, et al., (1982) EMBO J. 1:1589-1594 and Muller, et al., (1995) J. Plant Physiol 145:606-613); the 19 kDa zein promoter (Pedersen, et al., (1982) Cell 29:1019-1025); the 14 kDa zein promoter (Pedersen, et al., (1986) J. Biol. Chem. 261:6279-6284), the b-32 promoter (Lohmer, et al., (1991) EMBO J. 10:617-624); and the nitrite reductase (NiR) promoter (Rastogi, et al., (1997) Plant Mol. Biol. 34(3):465-76 and Sander, et al., (1995) Plant Mol. Biol. 27(1):165-77). For a review of consensus sequences found in nitrogen-induced promoters, see for example, Muller, et al., (1997) The Plant Journal 12:281-291.

Other useful promoters include F3.7 (U.S. Pat. No. 5,850,018) and the maize thioredoxin H promoter (Nu, et al., MGCNL 2004; U.S. Patent Application No. 60/514,123).

A promoter may fall into none, one, or more of the above groupings and may have utility in the present invention with respect to its tissue-specificity or timing or other characteristic, or with respect to a combination of such characteristics.

In addition, the constructs may contain control regions that regulate as well as engender expression. Generally, in accordance with many commonly practiced procedures, such regions will operate by controlling transcription, such as transcription factors, repressor binding sites and termination signals, among others. For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Transcription of the DNA encoding the polypeptides of the present invention by higher eukaryotes may be increased by inserting an enhancer sequence into the vector. Enhancers are cis-acting elements of DNA, usually about from 10 to 300 bp, that act to increase transcriptional activity of a promoter in a given host cell-type. Examples of enhancers include the SV40 enhancer, which is located on the late side of the replication origin at by 100 to 270, the cytomegalovirus early promoter enhancer, the polyoma enhancer on the late side of the replication origin, and adenovirus enhancers. Additional enhancers useful in the invention to increase transcription of the introduced DNA segment, include, inter alia, viral enhancers like those within the 35S promoter, as shown by Odell, et al., (1988) Plant Mol. Biol. 10:263-72, and an enhancer from an opine gene as described by Fromm, et al., (1989) Plant Cell 1:977. The enhancer may affect the tissue-specificity and/or temporal specificity of expression of sequences included in the vector.

Termination regions also facilitate effective expression by ending transcription at appropriate points. Useful terminators for practicing this invention include, but are not limited to, pinII (see, An, et al., (1989) Plant Cell 1(1):115-122), glb1 (see Genbank Accession Number L22345), gz (see, gzw64a terminator, Genbank Accession Number S78780), and the nos terminator from Agrobacterium.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway, et al., (1986) Biotechniques 5:320-335), electroporation (Riggs, et al., (1986) Proc. Natl. Acad. Sci. USA 83:5602-5606, Agrobacterium-mediated transformation (Townsend, et al., U.S. Pat. No. 5,563,055; Zhao, et al., U.S. Pat. No. 5,981,850), direct gene transfer (Paszkowski, et al., (1985) EMBO J. 3:2717-2722), and ballistic particle acceleration (see, for example, Sanford, et al., U.S. Pat. No. 5,955,050; Tomes, et al., U.S. Pat. No. 5,879,918; Tomes, et al., U.S. Pat. No. 5,886,255; Bidney, et al., U.S. Pat. No. 5,932,782; Tomes, et al., (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe, et al., (1988) Biotechnology 6:923-926); and Led transformation (WO 00/28058). Also see, Weissinger, et al., (1988) Ann. Rev. Genet. 22:521-577; Sanford, et al., (1987) Particulate Science and Technology 5:27-37 (onion); Christou, et al., (1988) Plant Physiol. 87:671-675 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-325 (soybean); Datta, et al., (1990) Biotechnology 8:736-750 (rice); Klein, et al., (1988) Proc. Natl. Acad. Sci. USA 85:5305-5309 (maize); Klein, et al., (1988) Biotechnology 6:559-563 (maize); Tomes, U.S. Pat. No. 5,250,855; Buising, et al., U.S. Pat. Nos. 5,322,783 and 5,325,656; Tomes, et al., (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein, et al., (1988) Plant Physiol. 91:550-555 (maize); Fromm, et al., (1990) Biotechnology 8:833-839 (maize); Hooykaas-Van Slogteren, et al., (1985) Nature (London) 311:763-765; Bowen, et al., U.S. Pat. No. 5,736,369 (cereals); Bytebier, et al., (1987) Proc. Natl. Acad. Sci. USA 85:5355-5359 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:515-518 and Kaeppler, et al., (1992) Theor. Appl. Genet. 85:560-566 (whisker-mediated transformation); D′Halluin, et al., (1992) Plant Cell 5:1595-1505 (electroporation); Li, et al., (1993) Plant Cell Reports 12:250-255 and Christou and Ford, (1995) Annals of Botany 75:507-513 (rice); Osjoda, et al., (1996) Nature Biotechnology 15:755-750 (maize via Agrobacterium tumefaciens); Leelavathi, et al., (2004) Plant Cell Reports 22:465-470 (cotton via Agrobacterium tumefaciens); Kumar, et al., (2004) Plant Molecular Biology 56:203-216 (cotton plastid via bombardment); all of which are herein incorporated by reference.

In specific embodiments, the type A RR sequences employed in the invention can be provided to a plant using a variety of transient transformation methods. Such transient transformation methods include, but are not limited to, the introduction of the type A RR protein or variants and fragments thereof directly into the plant or the introduction of the type A RR transcript into the plant. Such methods include, for example, microinjection or particle bombardment. See, for example, Crossway, et al., (1986) Mol. Gen. Genet. 202:179-185; Nomura, et al., (1986) Plant Sci. 55:53-58; Hepler, et al., (1995) Proc. Natl. Acad. Sci. 91:2176-2180 and Hush, et al., (1995) The Journal of Cell Science 107:775-785, all of which are herein incorporated by reference. Alternatively, the type A RR polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3153).

In other embodiments, the polynucleotide of the invention may be introduced into plants by contacting plants with a virus or viral nucleic acids. Generally, such methods involve incorporating a nucleotide construct of the invention within a viral DNA or RNA molecule. It is recognized that the a type A RR of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25855, WO99/25850, WO99/25855, and WO99/25853, all of which are herein incorporated by reference. Briefly, the polynucleotide of the invention can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-85. These plants may then be pollinated with either the same transformed strain or different strains, and the resulting progeny having desired expression of the phenotypic characteristic of interest can be identified. Two or more generations may be grown to ensure that expression of the desired phenotypic characteristic is stably maintained and inherited, and then seeds can be harvested to ensure expression of the desired phenotypic characteristic has been achieved. In this manner, the present invention provides a transformed seed (also referred to as a “transgenic seed”) having a polynucleotide of the invention, for example, an expression cassette of the invention, stably incorporated into its genome.

Pedigree breeding generally starts with the crossing of two genotypes, such as an elite line of interest and one other line having one or more desirable characteristics (e.g., having stably incorporated a polynucleotide of the invention, having a modulated activity and/or level of the polypeptide of the invention) which complements the elite line of interest. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection. Typically in the pedigree method of breeding, five or more successive filial generations of selfing and selection are practiced: F1→F2; F2→F3; F3→F5; F5→F₅, etc. After a sufficient amount of inbreeding, successive filial generations will serve to increase seed of the developed inbred. Preferably, the inbred line comprises homozygous alleles at about 95% or more of its loci.

In addition to being used to create a backcross conversion, backcrossing can also be used in combination with pedigree breeding to modify an elite line of interest and a hybrid that is made using the modified elite line. As discussed previously, backcrossing can be used to transfer one or more specifically desirable traits from one line, the donor parent, to an inbred called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, an F1, such as a commercial hybrid, is created. This commercial hybrid may be backcrossed to one of its parent lines to create a BC1 or BC2. Progeny are selfed and selected so that the newly developed inbred has many of the attributes of the recurrent parent and yet several of the desired attributes of the non-recurrent parent. This approach leverages the value and strengths of the recurrent parent for use in new hybrids and breeding.

Therefore, an embodiment of this invention is a method of making a backcross conversion of maize inbred line of interest, comprising the steps of crossing a plant of maize inbred line of interest with a donor plant comprising a mutant gene or transgene conferring a desired trait (i.e., increased root growth, increased yield, increased tolerance to drought, increased or maintained seed set during abiotic conditions, increased shoot growth, delayed senescence, or increased photosynthesis), selecting an F1 progeny plant comprising the mutant gene or transgene conferring the desired trait, and backcrossing the selected F1 progeny plant to the plant of maize inbred line of interest. This method may further comprise the step of obtaining a molecular marker profile of maize inbred line of interest and using the molecular marker profile to select for a progeny plant with the desired trait and the molecular marker profile of the inbred line of interest. In the same manner, this method may be used to produce an F1 hybrid seed by adding a final step of crossing the desired trait conversion of maize inbred line of interest with a different maize plant to make F1 hybrid maize seed comprising a mutant gene or transgene conferring the desired trait.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, selfed progeny and topcrossing. The selected progeny are cross-pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar. A synthetic cultivar is the resultant progeny formed by the intercrossing of several selected inbreds.

Mass selection is a useful technique when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program.

Mutation breeding is one of many methods that could be used to introduce new traits into an elite line. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays, Gamma rays (e.g., cobalt 60 or cesium 137), neutrons, (product of nuclear fission by uranium 235 in an atomic reactor), Beta radiation (emitted from radioisotopes such as phosphorus 32 or carbon 15), or ultraviolet radiation (preferably from 2500 to 2900 nm), or chemical mutagens (such as base analogues (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques, such as backcrossing. Details of mutation breeding can be found in “Principles of Cultivar Development” Fehr, 1993, Macmillan Publishing Company, the disclosure of which is incorporated herein by reference. In addition, mutations created in other lines may be used to produce a backcross conversion of elite lines that comprise such mutations.

The present invention may be used for transformation of any plant species, including, but not limited to, monocots and dicots. Examples of plant species of interest include, but are not limited to, corn (Zea mays, also known as maize), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus effiotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Typically, an intermediate host cell will be used in the practice of this invention to increase the copy number of the cloning vector. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:5057) and the lambda derived P L promoter and N-gene ribosome binding site (Shimatake, et al., (1981) Nature 292:128). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-235); Mosbach, et al., (1983) Nature 302:553-555).

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous polynucleotides in yeast is well known (Sherman, et al., (1982) Methods in Yeast Genetics, Cold Spring Harbor Laboratory). Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lists. The monitoring of the purification process can be accomplished by using Western blot techniques or radioimmunoassay of other standard immunoassay techniques.

The sequences of the present invention can also be ligated to various expression vectors for use in transfecting cell cultures of, for instance, mammalian, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:59), and necessary processing information sites, such as ribosome binding sites, RNA splice sites, polyadenylation sites (e.g., an SV50 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (see, Schneider, (1987) J. Embryol. Exp. Morphol. 27:353-365).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV50 (Sprague, et al., (1983) J. Virol. 55:773-781). Additionally, gene sequences to control replication in the host cell may be incorporated into the vector such as those found in bovine papilloma virus type-vectors (Saveria-Campo, (1985) DNA Cloning Vol. II a Practical Approach, D. M. Glover, Ed., IRL Press, Arlington, Va., pp. 213-238).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art (Kuchler, (1997) Biochemical Methods in Cell Culture and Virology, Dowden, Hutchinson and Ross, Inc.).

In certain embodiments the nucleic acid sequences of the present invention can be stacked with any combination of polynucleotide sequences of interest in order to create plants with a desired phenotype. The combinations generated may include multiple copies of any one of the polynucleotides of interest. For example, a polynucleotide of the present invention may be stacked with any other polynucleotide(s) of the present invention. The polynucleotides of the present invention can also be stacked with any other gene or combination of genes to produce plants with a variety of desired trait combinations including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106; and WO 98/20122); and high methionine proteins (Pedersen, et al., (1986) J. Biol. Chem. 261:6279; Kirihara, et al., (1988) Gene 71:359; and Musumura, et al., (1989) Plant Mol. Biol. 12:123)); increased digestibility (e.g., modified storage proteins (U.S. patent application Ser. No. 10/053,410, filed Nov. 7, 2001); and thioredoxins (U.S. patent application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility, stalk strength, flowering time, or transformation technology traits such as cell cycle regulation or gene targeting (e.g., WO 99/61619; WO 00/17364; WO 99/25821), the disclosures of which are herein incorporated by reference.

These stacked combinations can be created by any method including but not limited to cross breeding plants by any conventional or TopCross methodology, or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant.

II. Modulating the Concentration and/or Activity of a Type A Response Regulator Polypeptide

A method for modulating the concentration and/or activity of a polypeptide of the present invention in a plant is provided. In general, concentration and/or activity is increased or decreased by at least 1%, 5%, 10%, 20%, 30%, 50%, 50%, 60%, 70%, 80% or 90% relative to a native control plant, plant part, or cell. Modulation in the present invention may occur at any desired stage of development. In specific embodiments, the polypeptides of the present invention are modulated in monocots, particularly maize.

A “subject plant or plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The expression level of the type A RR polypeptide may be measured directly, for example, by assaying for the level of the type A RR polypeptide in the plant, or indirectly, for example, by measuring the response regulator activity of the type A RR polypeptide in the plant. Methods for determining the response regulator activity are described elsewhere herein and include evaluation of phenotypic changes, such as modulated shoot growth, seed set, callus growth with reduced cytokinins, or modulated root development, as well as molecular analyses such as effect on expression of cytokinin-responsive genes.

In specific embodiments, the RR polypeptide or polynucleotide employed in the invention is introduced into the plant cell. Subsequently, a plant cell having the introduced sequence is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and are discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,985; all of which are herein incorporated by reference. See also, WO 98/59350, WO 99/07865, WO 99/25821, and Beetham, et al., (1999) Proc. Natl. Acad. Sci. USA 96:8775-8778; herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprise at least one nucleotide.

A. Increasing the Activity and/or Level of a Response Regulator Polypeptide

Methods are provided to increase the activity and/or level of a type A RR polypeptide. An increase in the level and/or activity of the type A RR polypeptide of the invention can be achieved by providing to the plant a type A RR polypeptide. The type A RR polypeptide can be provided by introducing the amino acid sequence encoding the type A RR polypeptide into the plant, introducing into the plant a nucleotide sequence encoding a type A RR polypeptide, or alternatively, by modifying a genomic locus encoding the RR polypeptide.

As discussed elsewhere herein, many methods are known in the art for providing a polypeptide to a plant including, but not limited to, direct introduction of the polypeptide into the plant, introducing into the plant (transiently or stably) a polynucleotide construct encoding a polypeptide having response regulatory activity. It is also recognized that the methods of the invention may employ a polynucleotide that is not capable of directing, in the transformed plant, the expression of a protein or an RNA. Thus, the level and/or activity of a type A RR polypeptide may be increased by altering the gene encoding the type A RR polypeptide or its promoter. See, e.g., Kmiec, U.S. Pat. No. 5,565,350; Zarling, et al., PCT/US93/03868. Therefore mutagenized plants that carry mutations in type A RR genes, where the mutations increase expression of the type A RR gene or increase the response regulatory activity of the encoded type A RR polypeptide are provided.

B. Reducing the Activity and/or Level of a Type A RR Polypeptide

Methods are provided to reduce or eliminate the level and/or the activity of a type A RR polypeptide by transforming a plant cell with an expression cassette that expresses a polynucleotide that inhibits the expression of the type A RR polypeptide. The polynucleotide may inhibit the expression of one or more type A RR polypeptides directly, by preventing translation of the type A RR messenger RNA, or indirectly, by encoding a polypeptide that inhibits the transcription or translation of a plant gene encoding a type A RR polypeptide. Methods for inhibiting or eliminating the expression of a gene in a plant are well known in the art, and any such method may be used in the present invention to inhibit the expression of one or more type A RR polypeptide.

In accordance with the present invention, the expression of a type A RR polypeptide is inhibited if the protein level of the type A RR polypeptide is statistically significantly lower than the protein level of the same type A RR polypeptide in a plant that has not been genetically modified or mutagenized to inhibit the expression of that protein. In particular embodiments of the invention, the protein level of the type A RR polypeptide in a modified plant according to the invention is less than 96%, less than 90%, less than 80%, less than 75%, less than 60%, less than 50%, less than 50%, less than 30%, less than 20%, less than 10%, or less than 5% of the protein level of the same type A RR polypeptide in a plant that is not a mutant or that has not been genetically modified to inhibit the expression of that type A RR polypeptide. The expression level of the type A RR polypeptide may be measured directly, for example, by assaying for the level of type A RR polypeptide expressed in the plant cell or plant, or indirectly, for example, by measuring the response regulator activity of the type A RR polypeptide in the plant cell or plant. Methods for determining the response regulator activity of type A RR polypeptide are described elsewhere herein.

In other embodiments of the invention, the activity of one or more type A RR is reduced or eliminated by transforming a plant cell with an expression cassette comprising a polynucleotide encoding a polypeptide that inhibits the activity of one or more type A RR. The response regulator activity of a type A RR is inhibited according to the present invention if the response regulator activity of the type A RR is statistically significantly lower than the response regulator activity of the same type A RR in a plant that has not been genetically modified to inhibit the response regulator activity of that type A RR. In particular embodiments of the invention, the response regulator activity of the type A RR in a modified plant according to the invention is less than 95%, less than 90%, less than 80%, less than 70%, less than 60%, less than 50%, less than 50%, less than 30%, less than 20%, less than 10%, or less than 5% of the response regulator activity of the same type A RR in a plant that that has not been genetically modified to inhibit the expression of that type A RR. The response regulator activity of a type A RR is “eliminated” according to the invention when it is not detectable by the assay methods described elsewhere herein. Methods of determining the response regulator activity of a type A RR are described elsewhere herein.

In other embodiments, the activity of a type A RR may be reduced or eliminated by disrupting the gene encoding the type A RR. The invention encompasses mutagenized plants that carry mutations in type A RR genes, where the mutations reduce expression of the type A RR gene or inhibit the response regulator activity of the encoded type A RR.

Thus, many methods may be used to reduce or eliminate the activity of a type A RR. More than one method may be used to reduce the activity of a single type A RR. In addition, combinations of methods may be employed to reduce or eliminate the activity of two or more different type A RR polypeptides.

Non-limiting examples of methods of reducing or eliminating the expression of a type A RR are given below.

### 1. Polynucleotide-Based Methods

In some embodiments of the present invention, a plant cell is transformed with an expression cassette that is capable of expressing a polynucleotide that inhibits the expression of type A RR polypeptides. The term “expression” as used herein refers to the biosynthesis of a gene product, including the transcription and/or translation of said gene product. For example, for the purposes of the present invention, an expression cassette capable of expressing a polynucleotide that inhibits the expression of at least one type A RR polypeptide is an expression cassette capable of producing an RNA molecule that inhibits the transcription and/or translation of at least one type A RR polypeptide. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a type A RR polypeptide are given below.

i. Sense Suppression/Cosuppression

In some embodiments of the invention, inhibition of the expression of a type A RR polypeptide may be obtained by sense suppression or cosuppression. For cosuppression, an expression cassette is designed to express an RNA molecule corresponding to all or part of a messenger RNA encoding a type A RR polypeptide in the “sense” orientation. Over-expression of the RNA molecule can result in reduced expression of the native gene. Accordingly, multiple plant lines transformed with the cosuppression expression cassette are screened to identify those that show the greatest inhibition of type A RR polypeptide expression.

The polynucleotide used for cosuppression may correspond to all or part of the sequence encoding the type A RR polypeptide, all or part of the 5′ and/or 3′ untranslated region of a type A RR transcript, or all or part of both the coding sequence and the untranslated regions of a transcript encoding type A RR polypeptide. In some embodiments where the polynucleotide comprises all or part of the coding region for the type A RR polypeptide, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin, et al., (2002) Plant Cell 15:1517-1532. Cosuppression may also be used to inhibit the expression of multiple proteins in the same plant. Methods for using cosuppression to inhibit the expression of endogenous genes in plants are described in Flavell, et al., (1995) Proc. Natl. Acad. Sci. USA 91:3590-3596; Jorgensen, et al., (1996) Plant Mol. Biol. 31:957-973; Johansen and Carrington, (2001) Plant Physiol. 126:930-938; Broin, et al., (2002) Plant Cell 15:1517-1532; Stoutjesdijk, et al., (2002) Plant Physiol. 129:1723-1731; Yu, et al., (2003) Phytochemistry 63:753-763; and U.S. Pat. Nos. 5,035,323, and 5,283,185; each of which is herein incorporated by reference. The efficiency of cosuppression may be increased by including a poly-dT region in the expression cassette at a position 3′ to the sense sequence and 5′ of the polyadenylation signal. See, US Patent Publication Number 20020058815, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,185 and 5,035,323; herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing. (Aufsatz, et al., (2002) PNAS 99(4):16499-16506; Mette, et al., (2000) EMBO J. 19(19):5194-5201)

ii. Antisense Suppression

In some embodiments of the invention, inhibition of the expression of the type A RR polypeptide may be obtained by antisense suppression. For antisense suppression, the expression cassette is designed to express an RNA molecule complementary to all or part of a messenger RNA encoding the type A RR polypeptide. Over-expression of the antisense RNA molecule can result in reduced expression of the native gene. Accordingly, multiple plant lines transformed with the antisense suppression expression cassette are screened to identify those that show the greatest inhibition of type A RR polypeptide expression.

The polynucleotide for use in antisense suppression may correspond to all or part of the complement of the sequence encoding the type A RR polypeptide, all or part of the complement of the 5′ and/or 3′ untranslated region of the type A RR polypeptide transcript, or all or part of the complement of both the coding sequence and the untranslated regions of a transcript encoding the type A RR polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. Furthermore, portions of the antisense nucleotides may be used to disrupt the expression of the target gene. Generally, sequences of at least 50 nucleotides, 100 nucleotides, 200 nucleotides, 300, 500, 550, 500, 550 or greater may be used. Methods for using antisense suppression to inhibit the expression of endogenous genes in plants are described, for example, in Liu, et al., (2002) Plant Physiol. 129:1732-1753 and U.S. Pat. No. 5,759,829, which is herein incorporated by reference. Efficiency of antisense suppression may be increased by including a poly-dT region in the expression cassette at a position 3′ to the antisense sequence and 5′ of the polyadenylation signal. See, US Patent Publication Number 20020058815, herein incorporated by reference.

iii. Double-Stranded RNA Interference

In some embodiments of the invention, inhibition of the expression of a type A RR polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of type A RR polypeptide expression. Methods for using dsRNA interference to inhibit the expression of endogenous plant genes are described in Waterhouse, et al., (1998) Proc. Natl. Acad. Sci. USA 95:13959-13965, Liu, et al., (2002) Plant Physiol. 129:1732-1753, and WO 99/59029, WO 99/53050, WO 99/61631, and WO 00/59035; each of which is herein incorporated by reference.

iv. Hairpin RNA Interference and Intron-Containing Hairpin RNA Interference

In some embodiments of the invention, inhibition of the expression of one or more type A RR polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 5:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:5985-5990; Stoutjesdijk, et al., (2002) Plant Physiol. 129:1723-1731; and Waterhouse and Helliwell, (2003) Nat. Rev. Genet. 5:29-38. Methods for using hpRNA interference to inhibit or silence the expression of genes are described, for example, in Chuang and Meyerowitz, (2000) Proc. Natl. Acad. Sci. USA 97:5985-5990; Stoutjesdijk, et al., (2002) Plant Physiol. 129:1723-1731; Waterhouse and Helliwell, (2003) Nat. Rev. Genet. 5:29-38; Pandolfini, et al., BMC Biotechnology 3:7, and US Patent Publication Number 20030175965; each of which is herein incorporated by reference. A transient assay for the efficiency of hpRNA constructs to silence gene expression in vivo has been described by Panstruga, et al., (2003) Mol. Biol. Rep. 30:135-150, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 507:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 507:319-320; Wesley, et al., (2001) Plant J. 27:581-590; Wang and Waterhouse, (2001) Curr. Opin. Plant Biol. 5:156-150; Waterhouse and Helliwell, (2003) Nat. Rev. Genet. 5:29-38; Helliwell and Waterhouse, (2003) Methods 30:289-295, and US Patent Publication Number 20030180955, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00905, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for type A RR polypeptide). Methods of using amplicons to inhibit the expression of endogenous plant genes are described, for example, in Angell and Baulcombe, (1997) EMBO J. 16:3675-3685, Angell and Baulcombe, (1999) Plant J. 20:357-362, and U.S. Pat. No. 6,656,805, each of which is herein incorporated by reference.

vi. Ribozymes

In some embodiments, the polynucleotide expressed by the expression cassette of the invention is catalytic RNA or has ribozyme activity specific for the messenger RNA of type A RR polypeptide. Thus, the polynucleotide causes the degradation of the endogenous messenger RNA, resulting in reduced expression of the type A RR polypeptide. This method is described, for example, in U.S. Pat. No. 5,987,071, herein incorporated by reference.

vii. Small Interfering RNA or Micro RNA

In some embodiments of the invention, inhibition of the expression of one or more type A RR polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 525:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of type A RR polypeptide expression, the 22-nucleotide sequence is selected from a type A RR transcript sequence and contains 22 nucleotides of said type A RR polypeptide sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

### 2. Polypeptide-Based Inhibition of Gene Expression

In one embodiment, the polynucleotide encodes a zinc finger protein that binds to a gene encoding a type A RR polypeptide, resulting in reduced expression of the gene. In particular embodiments, the zinc finger protein binds to a regulatory region of a type A RR polypeptide gene. In other embodiments, the zinc finger protein binds to a messenger RNA encoding a type A RR polypeptide and prevents its translation. Methods of selecting sites for targeting by zinc finger proteins have been described, for example, in U.S. Pat. No. 6,553,252, and methods for using zinc finger proteins to inhibit the expression of genes in plants are described, for example, in US Patent Publication Number 20030037355; each of which is herein incorporated by reference.

### 3. Polypeptide-Based Inhibition of Protein Activity

In some embodiments of the invention, the polynucleotide encodes an antibody that binds to at least one type A RR polypeptide, and reduces the response regulator activity of the type A RR polypeptide. In another embodiment, the binding of the antibody results in increased turnover of the antibody-type A RR polypeptide complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

### 4. Gene Disruption

In some embodiments of the present invention, the activity of a type A RR polypeptide is reduced or eliminated by disrupting the gene encoding the type A RR polypeptide. The gene encoding the type A RR polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have reduced response regulator activity.

i. Transposon Tagging

In one embodiment of the invention, transposon tagging is used to reduce or eliminate the response regulator activity of one or more type A RR polypeptides. Transposon tagging comprises inserting a transposon within an endogenous type A RR polypeptide gene to reduce or eliminate expression of the type A RR polypeptide. “Type A RR gene” is intended to mean the gene that encodes a type A RR polypeptide according to the invention.

In this embodiment, the expression of one or more type A RR polypeptides is reduced or eliminated by inserting a transposon within a regulatory region or coding region of the gene encoding the type A RR polypeptide. A transposon that is within an exon, intron, 5′ or 3′ untranslated sequence, a promoter, or any other regulatory sequence of a type A RR gene may be used to reduce or eliminate the expression and/or activity of the encoded type A RR polypeptide.

Methods for the transposon tagging of specific genes in plants are well known in the art. See, for example, Maes, et al., (1999) Trends Plant Sci. 5:90-96; Dharmapuri and Sonti, (1999) FEMS Microbiol. Lett. 179:53-59; Meissner, et al., (2000) Plant J. 22:265-275; Phogat, et al., (2000) J. Biosci. 25:57-63; Walbot, (2000) Curr. Opin. Plant Biol. 2:103-107; Gai, et al., (2000) Nucleic Acids Res. 28:95-96; Fitzmaurice, et al., (1999) Genetics 153:1919-1928). In addition, the TUSC process for selecting Mu insertions in selected genes has been described in Bensen, et al., (1995) Plant Cell 7:75-85; Mena, et al., (1996) Science 275:1537-1550; and U.S. Pat. No. 5,962,765; each of which is herein incorporated by reference.

ii. Mutant Plants with Reduced Activity

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 253:572-581; Okubara, et al., (1995) Genetics 137:867-875; and Quesada, et al., (2000) Genetics 155:521-536; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See, McCallum, et al., (2000) Nat. Biotechnol. 18:555-557, herein incorporated by reference.

Mutations that impact gene expression or that interfere with the function (response regulator activity) of the encoded protein are well known in the art. Insertional mutations in gene exons usually result in null-mutants. Mutations in conserved residues are particularly effective in inhibiting the response regulator activity of the encoded protein. Such mutants can be isolated according to well-known procedures, and mutations in different type A RR loci can be stacked by genetic crossing. See, for example, Gruis, et al., (2002) Plant Cell 15:2863-2882.

In another embodiment of this invention, dominant mutants can be used to trigger RNA silencing due to gene inversion and recombination of a duplicated gene locus. See, for example, Kusaba, et al., (2003) Plant Cell 15:1555-1567.

The invention encompasses additional methods for reducing or eliminating the activity of one or more type A RR polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972 and 5,871,985; each of which are herein incorporated by reference. See also, WO 98/59350, WO 99/07865, WO 99/25821, and Beetham, et al., (1999) Proc. Natl. Acad. Sci. USA 96:8775-8778; each of which is herein incorporated by reference.

iii. Modulating the Stress Tolerance of a Plant

Methods are provided for the use of the type A RR sequences of the invention to modulate the tolerance of a plant to abiotic stress. In specific embodiments, methods are provided to increase or maintain seed set during abiotic stress episodes. During periods of stress (i.e., drought, salt, heavy metals, temperature, etc.) embryo development is often aborted. In maize, halted embryo development results in aborted kernels on the ear. Preventing this kernel loss will maintain yield. Accordingly, methods are provided to increase the stress resistance in a plant (i.e., an early developing embryo). Modulating the level and/or activity of a type A RR sequence of the invention can also modulate floral development during periods of stress, and thus methods are provided to maintain or improve the flowering process in plants under stress. In one method, a type A RR nucleotide sequence is introduced into the plant and the level and/or activity of the type A RR polypeptide is modulated, thereby maintaining or improving the tolerance of the plant under stress conditions. In other methods, the type A RR nucleotide construct introduced into the plant is stably incorporated into the genome of the plant.

Significant yield instability can occur as a result of unfavorable environments during the lag phase of seed development. During this period, seeds undergo dramatic changes in ultra structure, biochemistry, and sensitivity to environmental perturbation; yet demonstrate little change in growth (as characterized by dry mass accumulation). Two important events that occur during the lag phase are initiation and division of endosperm cells and amyloplasts (which are the sites for starch deposition). It has been demonstrated that during the lag phase (beginning at pollination and continuing to around 10-12 DAP [days after pollination] in maize) a dramatic increase in cytokinin concentration immediately precedes maximum rates of endosperm cell division and amyloplast formation, indicating that this hormone plays a central role in these processes and in what is called the ‘sink strength’ of the developing seed. Cytokinins have been demonstrated to play an important role in establishing seed size, decreasing tip kernel abortion, and increasing seed set during unfavorable environmental conditions.

Methods are therefore provided to modulate the activity and/or level of the type A RR polypeptides in the developing female inflorescence, thereby elevating effective cytokinin levels and allowing developing seed to achieve their full genetic potential for size, minimize tip kernel abortion, and buffer seed set during unfavorable environments. The methods further allow the plant to maintain and/or improve the flowering process during unfavorable environments. These methods may include transformation with constructs designed to down-regulate expression of a type A RR polypeptide by any means, such as those described elsewhere herein.

In this embodiment, a variety of promoters could be used to direct the expression of a sequence capable of modulating the level and/or activity of the type A RR polypeptide. In one method, a promoter that is stress insensitive and is expressed in a tissue of the developing seed during the lag phase of development is used. By “insensitive to stress” is intended that the expression level of a sequence operably linked to the promoter is not altered or only minimally altered under stress conditions. By “lag phase” promoter is intended a promoter that is active in the lag phase of seed development. A description of this developmental phase is found elsewhere herein. By “developing seed-preferred” is intended a promoter that allows for enhanced expression within a developing seed (i.e., kernel). Such promoters that are stress insensitive and are expressed in a tissue of the developing seed during the lag phase of development are known in the art and include Zag2.1 (Theissen, et al., (1995) Gene 156:155-166, Genbank Accession Number X80206), and mzE40 (Zm40) (U.S. Pat. No. 6,403,862 and WO01/2178). Other promoters of interest include stress inducible promoters and promoters that are preferentially expressed in the developing kernel or immature ear tissue. Representative seed-preferred promoters, kernel-preferred promoter, immature ear tissue-preferred promoter, and inflorescense promoters are described elsewhere and herein.

Methods to assay for a modulation in seed set during abiotic stress are known in the art. For example, plants having the modulated type A RR activity can be monitored under various stress conditions and compared to controls plants. For instance, the plant having the modulated type A RR activity and/or level can be subjected to various degrees of stress during flowering and seed set. Under identical conditions, the genetically modified plant having the modulated level and/or activity of type A RR polypeptide will have a higher number and/or mass of developing kernels than a wild type (non-transformed) plant.

Accordingly, the present invention further provides plants having increased yield or a maintained yield during periods of abiotic stress (i.e., drought, salt, heavy metals, temperature, etc). In some embodiments, the plants having an increased or maintained yield during abiotic stress have a modulated level/activity of a type A RR polypeptide of the invention. In other embodiments, the plant comprises a type A RR nucleotide sequence of the invention operably linked to a promoter that drives expression in the plant cell. In other embodiments, such plants have stably incorporated into their genome a nucleic acid molecule comprising a type A RR nucleotide sequence of the invention operably linked to a promoter that drives expression in the plant cell.

iv. Modulating Shoot and Leaf Development

Methods are also provided for modulating shoot and leaf development in a plant. By “modulating shoot development” and/or “modulating leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length, and leaf senescence. As used herein, “leaf development” and “shoot development” encompass all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10587-10592 and US Patent Application Number 2003/0075698, each of which is herein incorporated by reference.

The method for modulating shoot and/or leaf development in a plant comprises modulating the activity and/or level of a type A RR polypeptide of the invention. In one embodiment, a type A RR sequence of the invention is provided. In other embodiments, the type A RR nucleotide sequence can be provided by introducing into the plant a polynucleotide comprising a type A RR nucleotide sequence of the invention, expressing the type A RR sequence, and thereby modifying shoot and/or leaf development. In other embodiments, the type A RR nucleotide construct introduced into the plant is stably incorporated into the genome of the plant.

In specific embodiments, shoot and/or leaf development is modulated by modulating the level and/or activity of the type A RR in the plant. A modulation in type A RR activity can result in at least one or more of the following alterations in shoot and/leaf development including, but not limited to, altered (increased or decreased) shoot growth, altered photosynthesis, modulated leaf number, altered leaf surface, altered length of internodes, and modulated leaf senescence. Modulating the level of the type A RR polypeptide in the plant can thereby increase plant yields.

As discussed above, one of skill will recognize the appropriate promoter to use to modulate shoot and leaf development of the plant. Exemplary promoters for this embodiment include constitutive promoters or promoters that are preferentially active in photosynthetic tissues including, for example, shoot-preferred promoters, shoot meristem-preferred promoters, and leaf-preferred promoters. Exemplary promoters have been disclosed elsewhere herein.

Accordingly, the present invention further provides plants having a modulated shoot and/or leaf development when compared to a control plant. In some embodiments, the plant of the invention has an increased level/activity or a decreased level/activity of a type A RR polypeptide of the invention.

In still other embodiments, methods are provided for modulating (enhancing or decreasing) shoot regeneration in callus. By “modulating shoot regeneration” is intended any alteration in shoot regeneration when compared to a control. Such alterations include, but are not limited to, an increase or decrease in the mean number of shoots per piece of callus; an increase or decrease in the frequency of shoot regenerating callus; and/or, an increase or decrease in the level or rate of shoot formation in the presence of lower concentrations of plant growth regulators. Methods to assay for such modulations in shoot regeneration are known. See, for example, Bahieldin, et al., (2000) Plant Breeding 119:537-539 and Popescu, et al., (2000) Acta Hort. (ISHS) 538:667-670, both of which are herein incorporated by reference.

Methods for modulating shoot regeneration in callus comprise modulating the level and/or activity of the type A RR polypeptide in the plant. In one method, a type A RR sequence of the invention is provided to the plant. In another method, the type A RR nucleotide sequence is provided by introducing into the plant a polynucleotide comprising a type A RR nucleotide sequence of the invention, expressing the type A RR sequence, and thereby modifying shoot regeneration from callus. In still other methods, the type A RR nucleotide construct introduced into the plant is stably incorporated into the genome of the plant. In one embodiment, the type A RR sequence of interest can be introduced into the plant, and subsequently, callus formed from the transgenic plant. Alternatively, the type A RR sequence could be introduced into the explant or callus, prior to shoot regeneration.

As discussed above, one of skill will recognize the appropriate promoter to use to modulate shoot regeneration. Exemplary promoters for this embodiment include shoot-preferred promoters, which have been disclosed elsewhere herein.

In still other embodiments, methods for modulating the responsiveness of a callus to a cytokinin is provided. In this method, modulating the level and/or activity of the type A RR will enhance the sensitivity of the plant to cytokinins. For example, use of methods to reduce expression of ZmRR5 in maize callus may result in increased sensitivity to exogneous cytokinin. Accordingly, lower concentrations of growth regulators (i.e., cytokinins) or no exogenous cytokinins in the culture medium will be needed to enhance shoot regeneration in callus.

Methods for establishing callus from explants are known. For example, roots, stems, buds, immature embryos and aseptically germinated seedlings are just a few of the sources of tissue that can be used to induce callus formation. Generally, young and actively growing tissues (i.e., young leaves, roots, meristems) are used, but are not required. Callus formation is controlled by growth regulating substances present in the medium (auxins and cytokinins). The specific concentrations of plant regulators needed to induce callus formation vary from species to species and can even depend on the source of explant. In some instances, it is advised to use different growth substances (i.e., 2,5-D or NAA) or a combination of them during tests, since some species may not respond to a specific growth regulator. In addition, culture conditions (i.e., light, temperature, etc.) can also influence the establishment of callus. Once established, callus cultures can be used to initiate shoot regeneration. See, for example, Gurel, et al., (2001) Turk J. Bot. 25:25-33; Dodds, et al., (1995). Experiments in Plant Tissue Culture, Cambridge University Press; Gamborg, (1995) Plant Cell, Tissue and Organ Culture, eds. G. Phillips; and, US Patent Application Publication Number 20030180952, all of which are herein incorporated by reference.

It is further recognized that increasing seed size and/or weight can also be accompanied by an increase in the rate of growth of seedlings or an increase in early vigor. In addition, modulating the plant's tolerance to stress, as discussed above, along with modulation of root, shoot and leaf development can increase plant yield and vigor. As used herein, the term “vigor” refers to the ability of a plant to grow rapidly during early development, and relates to the successful establishment, after germination, of a well-developed root system and a well-developed photosynthetic apparatus. In addition, an increase in seed size and/or weight can also result in an increase in plant yield when compared to a control.

v. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development, or radial expansion.

The methods for modulating root development comprise modulating (reducing or increasing) the level and/or activity of the type A RR polypeptide in the plant. In one method, a type A RR nucleotide sequence is introduced into the plant and the level and/or activity of the type A RR polypeptide is modulated. In other methods, the type A RR nucleotide construct introduced into the plant is stably incorporated into the genome of the plant.

A modulation in type A RR activity can result in at least one or more of the following alterations to root development, including, but not limited to, larger root meristems, increased root growth, enhanced radial expansion, an enhanced vasculature system, increased root branching, more adventitious roots, and/or increased fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc. Methods of measuring such developmental alterations in the root system are known in the art. See, for example, US Patent Application Publication Number 2003/0075698 and Werner, et al., (2001) PNAS 18:10587-10592, both of which are herein incorporated by reference.

As discussed above, one of skill will recognize the appropriate promoter to use to modulate root development in the plant. Exemplary promoters for this embodiment include root-preferred promoters, which have been disclosed elsewhere herein.

Stimulating root growth and increasing root mass by modulating the activity and/or level of the polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by modulating the level and/or activity of the type A RR polypeptide also finds use in promoting in vitro propagation of explants.

Accordingly, the present invention further provides plants having modulated root development when compared to the root development of a control plant. In some embodiments, the plant of the invention has a modulated level/activity of the type A RR polypeptide of the invention and has enhanced root growth and/or root biomass. In other embodiments, such plants have stably incorporated into their genome a nucleic acid molecule comprising a type A RR nucleotide sequence of the invention operably linked to a root-preferred promoter that drives expression in the plant cell, wherein expression of the sequence modulates the level and/or activity of the type A RR polypeptide.

vi. Modulating Responsiveness to Cytokinin

As used herein a “cytokinin” refers to a class of plant-specific hormones that play a central role during the cell cycle and influence numerous developmental programs. Cytokinins comprise an N⁶-substituted purine derivative. Representative cytokinins include isopentenyladenine (N⁶-(Δ²-isopentenyl) adenine (hereinafter, iP), zeatin (6-(5-hydroxy-3-methylbut-trans-2-enylamino) purine) (hereinafter, Z), dihydrozeatin (DZ) and benzyladenine (BA). The free bases and their ribosides (iPR, ZR, and DZR) are believed to be the active compounds. Additional cytokinins are known. See, for example, U.S. Pat. No. 5,211,738, herein incorporated by reference.

Type A RR may be involved in the transcriptional activator cascade of cytokinin signaling. Therefore, modulating the levels of type A RR polypeptides may modulate the level/activity of cytokinin. “Modulating the level and/or activity of cytokinin” includes any decrease or increase in cytokinin level and/or activity in the plant, including an altered responsiveness to cytokinin. For example, modulating the level and/or activity can comprise either an increase or a decrease in overall cytokinin level/activity of about 0.1%, 0.5%, 1%, 3% 5%, 10%, 15%, 20%, 25%, 30%, 35%, 50%, 55%, 50%, 55%, 60%, 65%, 70%, 75%, 80%, 85%, 90%, 95% or greater when compared to a control plant or plant part. Alternatively, the modulated level and/or activity of the cytokinin can include about a 0.5 fold, 1 fold, 2 fold, 5 fold, 8 fold, 16 fold or 32 fold change in cytokinin level/activity in the plant or a plant part when compared to a control plant or plant part.

It is further recognized that the modulation of the cytokinin level/activity need not be an overall increase/decrease in cytokinin level and/or activity, but also includes a change in tissue distribution of the cytokinin. See, for example, Jones, et al., (1997) Plant Growth Regul 23:123-135, Turner, et al., (1985) Plant Physiol 79:321-322, and Mok, et al., (2001) Annu Rev Plant Physiol Plant Mol Biol 52:89-118, each of which are herein incorporated by reference.

Moreover, the modulation of the cytokinin level/activity need not be an overall increase/decrease in cytokinins, but also includes a change in the ratio of various cytokinin derivatives. For example, the ratio of various cytokinin derivatives such as isopentenyladenine-type, zeatin-type, or dihydrozeatin-type cytokinins, and the like, could be altered and thereby modulate the level/activity of the cytokinin of the plant or plant part when compared to a control plant.

Methods for assaying for a modulation in cytokinin level and/or activity are known in the art. For example, representative methods for cytokinin extraction, immunopurification, HPLC separation, and quantification by ELISA methods can be found, for example, in Faiss, et al., (1997) Plant J. 12:501-515. See, also, Werner, et al., (2001) PNAS 98:10587-10592) and Dewitte, et al., (1999) Plant Physiol. 119:111-121. Each of these references are herein incorporated by reference.

In specific methods, the level and/or activity of a cytokinin in a plant is modulated by increasing the level or activity of the type A RR polypeptide in the plant. Methods for increasing the level and/or activity of type A RR polypeptides in a plant are discussed elsewhere herein. Briefly, such methods comprise providing a type A RR polypeptide of the invention to a plant and thereby increasing the level and/or activity of the type A RR polypeptide. In other embodiments, a type A RR nucleotide sequence encoding a type A RR polypeptide can be provided by introducing into the plant a polynucleotide comprising a type A RR nucleotide sequence of the invention, expressing the type A RR sequence, increasing the activity of the type A RR polypeptide, and thereby modulating the level and/or activity of a cytokinin in the plant or plant part. In other embodiments, the type A RR nucleotide construct introduced into the plant is stably incorporated into the genome of the plant.

In other methods, the level and/or activity of a cytokinin in a plant is modulated by decreasing the level and/or activity of the type A RR polypeptide in the plant. Such methods are disclosed in detail elsewhere herein. In one such method, a type A RR nucleotide sequence is introduced into the plant and expression of said type A RR nucleotide sequence decreases the activity of the type A RR polypeptide, and thereby modulates the level and/or activity of a cytokinin in the plant or plant part. In other embodiments, the type A RR nucleotide construct introduced into the plant is stably incorporated into the genome of the plant.

As discussed above, one of skill will recognize the appropriate promoter to use to modulate the level/activity of a cytokinin in the plant. Exemplary promoters for this embodiment have been disclosed elsewhere herein.

Accordingly, the present invention further provides plants having a modulated level/activity of a cytokinin when compared to the cytokinin level/activity of a control plant. In one embodiment, the plant of the invention having a modulated level/activity of cytokinin has an increased level/activity of the type A RR polypeptide of the invention or alternatively has a reduced or eliminated level of the type A RR polypeptide of the invention. In other embodiments, such plants have stably incorporated into their genome a nucleic acid molecule comprising a type A RR nucleotide sequence of the invention operably linked to a promoter that drives expression in the plant cell.

As demonstrated below, expression of the type A RR polypeptides is modulated in the presence of cytokinin. Accordingly, the type A RR sequences of the invention find use as molecular markers to detect the presence or an alteration in the level of cytokinin. In addition, the type A RR sequences can also be used as molecular markers to detect the activity of other proteins in the cytokinin signaling or biosynthetic pathways. It is recognized such proteins could be either endogenous to the plant or heterologous to the plant. Methods to assay for the expression of the type A RR polypeptides are known in the art and include, but are not limited to, Northern analysis, RNase protection, or Western analysis.

In other methods of the invention, the level and/or activity of the type A RR polypeptide and the activity and/or level of at least one other polypeptide involved in cytokinin sensing or production is also modulated. For example, compositions and methods are provided that modulate the level and/or activity of a type A RR polypeptide and an isopentenyl transferase-like (IPT-like) protein. Such IPT and IPT-like sequences are described, for example, in U.S. patent application Ser. No. 11/228,659, filed Sep. 16, 2005, herein incorporated by reference in its entirety. Such methods and compositions find use in modulating cytokinin production and sensing in a plant.

The following examples are offered by way of illustration and not by way of limitation.

### EXPERIMENTAL

### Example 1

### Expression Patterns of ZmRR5 and ZmRR6 in Response To Cytokinin

Cytokinins are known to promote endosperm cell division and to play an important role in controlling kernel sink-strength. Active cytokinin pools are regulated by the rate of synthesis, storage, and/or degradation. Cytokinin degradation in maize is catalyzed by the enzyme cytokinin oxidase. The expression pattern of a gene encoding a cytokinin oxidase from maize (Ckx1) has been previously characterized. It was demonstrated that Ckx1 expression correlates with the natural accumulation of cytokinins during kernel development. Moreover, Ckx1 is induced ˜60 fold in maize leaf discs incubated with benzyladenine (BA) compared to untreated controls, and increased 3 to 5 fold in 5 DAP kernels cultured in vitro with BA (Brugière, et al., (2003) Plant Physiology 132:1228-1240). Thus in maize, cytokinin oxidase is a good reporter of elevated cytokinin levels.

It has also been shown that induction of Ckx1 by BA requires protein synthesis, which suggests de novo synthesis of a specific transcriptional regulator. Induction of Ckx1 may involve a response regulator pathway. This pathway typically consists of a histidine kinase receptor, histidine phosphotransfer proteins (ZmHPs), and two types of response regulators called type A and B. Several maize response regulators (ZmRR) are present in the public database and are differentially regulated by cytokinins (Sakakibara H, personal communication). Therefore learning how these maize response regulator genes respond to increased BA levels is of interest.

A Lynx experiment was performed to identify genes in the cytokinin signaling and metabolism pathway, as well as the carbohydrate pathway, whose expression is modulated in response to elevated cytokinin levels. One goal of this experiment was to identify response regulators that could be involved in the cytokinin-signaling pathway and could potentially act as Ckx1 transcriptional regulators.

Two experiments were performed: one was an initial experiment to determine the time course for BA induction of Ckx1 transcripts in leaf discs, while the second experiment used the time-course information to optimize tissue harvest for the actual Lynx study. For the time-course experiment, leaf discs (5 mm in diameter) were collected from fully expanded leaves of 8-week old maize plants (inbred B73) and were incubated in petri dishes containing water or water plus 10 μM BA for different time periods at 25° C. For the Lynx experiment ˜300 leaf-discs were punched from ear leaves collected from 5 different maize B73 plants at flowering. Half of the discs were floated in a large petri dish on a solution containing 10 μM BA; the other half were floated on distilled water. Discs were incubated for 6 h at 25° C. in the light, blotted-dried, frozen in liquid nitrogen, and then submitted for Lynx analysis.

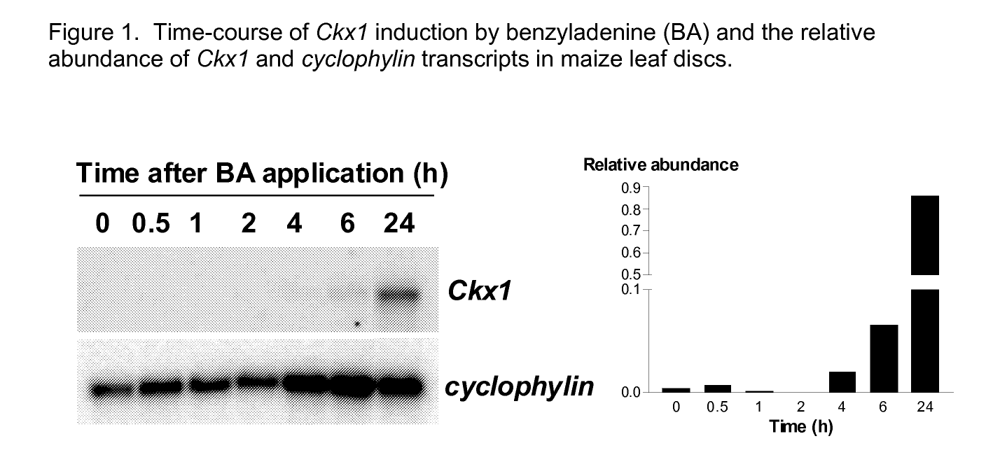

Time-course experiment: This perfunctory experiment was designed to learn the kinetics of Ckx1 transcript accumulation in response to BA. Ckx1 transcript levels were induced ˜15 fold after 6 hours (FIG. 1). This time-point was chosen for the Lynx study because it corresponds to the earliest significantly detectable response of Ckx1 expression to BA. 3 pg of polyA enriched RNA was used at each time point shown in FIG. 1.

Lynx experiment: Overall, differences in gene expression were found to be modest. Nevertheless, differences in levels of expression of multiple genes were detected and these are presented as genes relevant to cytokinin response, isoprenoid (or terpenoid) biosynthesis, and cell division (Table 2).

### A. Cytokinin Responsive, Cell Division and Chlorophyll Biosynthesis Genes:

A ˜30-fold increase in Ckx1 transcripts was measured after 6 h of BA application (Table 2). This result compares favorably with the 15-fold induction observed via our Northern blot in the perfunctory experiment (FIG. 1). We also identified two new response regulators whose expression is induced by BA, ZmRRS and ZmRR6 (Table 2).

TABLE 2
Expression pattern of selected genes involved in cytokinin response,
isoprenoid or terpenoid biosynthesis and cell division. ppm ratio
between control (Cldctl) and treated (Cldba) samples are shown. The
ratio change of up regulated genes and down regulated genes are shown.

| | | | Ratio | Up/down |
|---|---|---|---|---|
| Cldctl | Cldba | Best BLAST assignment | BA/Ctrl | regulation |
| 47 | 395 | Zm Response regulator 5 (ZmRR5) | 8.50 | up |
| 8 | 70 | Zm response regulator 6 (ZmRR6) | 8.75 | up |

### Example 2

### Modulating Seed Set During Stress

Immature maize embryos from greenhouse donor plants are bombarded with a plasmid containing an expression cassette designed to downregulate the maize RR5 sequence (SEQ ID NO: 1), as detailed in methods described elsewhere herein. The ZmRR5-specific polynucleotide is operably linked to a Zea mays RAB17 promoter and the selectable marker gene PAT (Wohlleben, et al., (1988) Gene 70:25-37), which confers resistance to the herbicide Bialaphos. Alternatively, the selectable marker gene is provided on a separate plasmid. Transformation is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 5 hours and then aligned within the 2.5 cm target zone in preparation for bombardment.

A plasmid vector comprising the maize RR5 sequence operably linked to a Zea mays RAB17 promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #5 in particle gun #HE35-1 or #HE35-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-5 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored under various stress conditions and compared to controls plants. A modulation in seed set during an abiotic stress episode will be monitored.

Bombardment medium (560Y) comprises 5.0 g/l N6 basal salts (SIGMA C-1516), 1.0 ml/l Eriksson's Vitamin Mix (1000×SIGMA-1511), 0.5 mg/l thiamine HCl, 120.0 g/l sucrose, 1.0 mg/l 2,5-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 5.0 g/l N6 basal salts (SIGMA C-1516), 1.0 ml/l Eriksson's Vitamin Mix (1000×SIGMA-1511), 0.5 mg/l thiamine HCl, 30.0 g/l sucrose, and 2.0 mg/l 2,5-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 5.3 g/l MS salts (GIBCO 11117-075), 5.0 ml/l MS vitamins stock solution (0.100 g nicotinic acid, 0.02 g/l thiamine HCL, 0.10 g/l pyridoxine HCL, and 0.50 g/l glycine brought to volume with polished

D-I H₂O) (Murashige and Skoog, (1962) Physiol. Plant. 15:573), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 5.3 g/l MS salts (GIBCO 11117-075), 5.0 ml/l MS vitamins stock solution (0.100 g/l nicotinic acid, 0.02 g/l thiamine HCL, 0.10 g/l pyridoxine HCL, and 0.50 g/l glycine brought to volume with polished D-I H₂O), 0.1 g/l myo-inositol, and 50.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 3

### Modulating Plant Yields

For Agrobacterium-mediated transformation of maize with the maize RR6 nucleotide sequence (SEQ ID NO: 4) operably linked to a Zea mays ubiquitin promoter, the method of Zhao is employed (U.S. Pat. No. 5,981,850, and PCT Patent Publication Number WO98/32326; the contents of which are hereby incorporated by reference). Briefly, immature embryos are isolated from maize and the embryos contacted with a suspension of Agrobacterium, where the bacteria are capable of transferring the RR6 nucleotide sequence to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 5: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

The plants are monitored for a modulation in shoot growth, leaf senescence, and/or photosynthesis when compared to an appropriate control plant. A modulation in plant yield is also monitored.

### Example 4

### Modulating Root Growth

Immature maize embryos from greenhouse donor plants are bombarded with a plasmid designed to achieve post-transcriptional gene silencing (PTGS) with an appropriate promoter. For example, the CRWAQ81 based root-preferred promoter could be employed. The plasmid comprises the CRWAQ81 promoter operably linked to a hairpin structure made from the CDS of the RR5 polynucleotide (SEQ ID NO: 1). The plasmid also contains the selectable marker gene PAT (Wohlleben, et al., (1988) Gene 70:25-37), which confers resistance to the herbicide Bialaphos. Transformation is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 5 hours and then aligned within the 2.5 cm target zone in preparation for bombardment.

A plasmid vector comprising the ZmRR5 sequence operably linked to a CRAWQ81 promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #5 in particle gun #HE35-1 or #HE35-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-5 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored under various stress conditions and compared to controls plants.

Plants are monitored and scored for a modulation in root development. The modulation in root development includes monitoring for a modulation in root growth of one or more root parts including the primary root, lateral roots, adventitious roots, etc. Methods of measuring such developmental alterations in the root system are known in the art. See, for example, US Patent Application Publication Number 2003/0075698 and Werner, et al., (2001) PNAS 18:10587-10592, both of which are herein incorporated by reference.

Bombardment medium (560Y) comprises 5.0 g/l N6 basal salts (SIGMA C-1516), 1.0 ml/l Eriksson's Vitamin Mix (1000×SIGMA-1511), 0.5 mg/l thiamine HCl, 120.0 g/l sucrose, 1.0 mg/l 2,5-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 5.0 g/l N6 basal salts (SIGMA C-1516), 1.0 ml/l Eriksson's Vitamin Mix (1000×SIGMA-1511), 0.5 mg/l thiamine HCl, 30.0 g/l sucrose, and 2.0 mg/l 2,5-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 5.3 g/l MS salts (GIBCO 11117-075), 5.0 ml/l MS vitamins stock solution (0.100 g nicotinic acid, 0.02 g/l thiamine HCL, 0.10 g/l pyridoxine HCL, and 0.50 g/l glycine brought to volume with polished D-I H₂O) (Murashige and Skoog, (1962) Physiol. Plant. 15:573), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 5.3 g/l MS salts (GIBCO 11117-075), 5.0 ml/l MS vitamins stock solution (0.100 g/l nicotinic acid, 0.02 g/l thiamine HCL, 0.10 g/l pyridoxine HCL, and 0.50 g/l glycine brought to volume with polished D-I H₂O), 0.1 g/1 myo-inositol, and 50.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 5

### Modulating Shoot Regeneration in Callus

For Agrobacterium-mediated transformation of maize with the maize RR6 nucleotide sequence (SEQ ID NO: 4) operably linked to a Zea mays ubiquitin promoter, the method of Zhao is employed (U.S. Pat. No. 5,981,850, and PCT patent publication WO98/32326; the contents of which are hereby incorporated by reference). Briefly, immature embryos are isolated from maize and the embryos contacted with a suspension of Agrobacterium, where the bacteria are capable of transferring the RR6 nucleotide sequence to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step), which may take place on solid medium. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Next, inoculated embryos are cultured on medium containing a selective agent and growing transformed callus is recovered (step 4: the selection step). As the callus is then regenerated into plants on solid medium (step 5: the regeneration step), callus tissue and plants are monitored for a modulation of shoot or root growth, responsiveness to exogenous hormone concentrations, and/or a modulation in overall vigor when compared to an appropriate control plant.

### Example 6

### Soybean Transformation

Soybean embryos are bombarded with a plasmid containing the maize RR5 sequence operably linked to a Zea mays ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 5,955,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188), and the 3′ region of the nopaline synthase gene from the T-DNA of the Ti plasmid of Agrobacterium tumefaciens. The expression cassette comprising the RR5 operably linked to the Zea mays ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 500 μl 70% ethanol and resuspended in 50 μl of anhydrous ethanol. The DNA/particle suspension can be sonicated three times for one second each. Five microliters of the DNA-coated gold particles are then loaded on each macro carrier disk.

Approximately 300-500 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 7

### Sunflower Meristem Tissue Transformation

Sunflower meristem tissues are transformed with an expression cassette containing the RR6 sequence (SEQ ID NO: 4) operably linked to a Zea mays ubiquitin promoter as follows (see also, European Patent Number EP 0 586233, herein incorporated by reference, and Malone-Schoneberg, et al., (1995) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant. 15:573-597), Shepard's vitamin additions (Shepard, (1980) in Emergent Techniques for the Genetic Improvement of Crops (University of Minnesota Press, St. Paul, Minn.), 50 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 5.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS1000® particle acceleration device.

Disarmed Agrobacterium tumefaciens strain EHA105 is used in all transformation experiments. A binary plasmid vector comprising the expression cassette that contains the RR6 gene operably linked to the Zea mays ubiquitin promoter is introduced into Agrobacterium strain EHA105 via freeze-thawing as described by Holsters, et al., (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.5 to 0.8. The Agrobacterium cells are pelleted and resuspended at a final 0D₆₀₀ of 0.5 in an inoculation medium comprised of 12.5 mM MES pH 5.7, 1 gm/l NH₅Cl, and 0.3 gm/l MgSO₅.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 375B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 375B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for response regulator activity.

NPTII-positive shoots are grafted to Pioneer® hybrid 6550 in vitro-grown sunflower seedling rootstock. Surface sterilized seeds are germinated in 58-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by response regulator activity analysis of leaf extracts while transgenic seeds harvested from NPTII-positive T₀ plants are identified by response regulator activity analysis of small portions of dry seed cotyledon.

### Example 8

### Rice Transformation

One method for transforming DNA into cells of higher plants that is available to those skilled in the art is high-velocity ballistic bombardment using metal particles coated with the nucleic acid constructs of interest (see, Klein, et al., (1987) Nature (London) 327:70-73, and see, U.S. Pat. No. 4,945,050). A Biolistic PDS-1000/He (BioRAD Laboratories, Hercules, Calif.) is used for these complementation experiments.

The bacterial hygromycin B phosphotransferase (Hpt II) gene from Streptomyces hygroscopicus that confers resistance to the antibiotic may be used as the selectable marker for rice transformation. In the vector, the Hpt II gene may be engineered with the 35S promoter from Cauliflower Mosaic Virus and the termination and polyadenylation signals from the octopine synthase gene of Agrobacterium tumefaciens. For example, see the description of vector pML18 in WO 97/47731, published on Dec. 18, 1997, the disclosure of which is hereby incorporated by reference.

Embryogenic callus cultures derived from the scutellum of germinating rice seeds serve as source material for transformation experiments. This material is generated by germinating sterile rice seeds on a callus initiation media (MS salts, Nitsch and Nitsch vitamins, 1.0 mg/1 2,4-D and 10 μM AgNO₃) in the dark at 27-28° C. Embryogenic callus proliferating from the scutellum of the embryos is transferred to CM media (N6 salts, Nitsch and Nitsch vitamins, 1 mg/1 2,4-D, Chu, et al., (1985) Sci. Sinica 18:659-668). Callus cultures are maintained on CM by routine sub-culture at two-week intervals and used for transformation within 10 weeks of initiation.

Callus is prepared for transformation by subculturing 0.5-1.0 mm pieces approximately 1 mm apart, arranged in a circular area of about 4 cm in diameter, in the center of a circle of Whatman #541 paper placed on CM media. The plates with callus are incubated in the dark at 27-28° C. for 3-5 days. Prior to bombardment, the filters with callus are transferred to CM supplemented with 0.25 M mannitol and 0.25 M sorbitol for 3 hr in the dark. The petri dish lids are then left ajar for 20-45 minutes in a sterile hood to allow moisture on tissue to dissipate.

Each genomic DNA fragment is co-precipitated with pML18 (containing the selectable marker for rice transformation) onto the surface of gold particles. To accomplish this, a total of 10 μg of DNA at a 2:1 ratio of trait:selectable marker DNAs are added to 50 μl aliquot of gold particles that have been resuspended at a concentration of 60 mg ml⁻¹. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are washed twice with 1 ml of absolute ethanol and then resuspended in 50 μl of absolute ethanol and sonicated (bath sonicator) for one second to disperse the gold particles. The gold suspension is incubated at −70° C. for five minutes and sonicated (bath sonicator) if needed to disperse the particles. Six μl of the DNA-coated gold particles are then loaded onto mylar macrocarrier disks and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen and the callus is bombarded two times. Two to four plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/l hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipet. Three ml aliquots of the callus suspension are plated onto fresh SM media and the plates are incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% gelrite+50 ppm hyg B) for 2 weeks in the dark at 25° C. After 2 weeks the callus is transferred to RM2 media (MS salts, Nitsch and Nitsch vitamins, 3% sucrose, 0.4% gelrite+50 ppm hyg B) and placed under cool white light (˜40 μm⁻²s⁻¹) with a 12 hr photoperiod at 25° C. and 30-40% humidity. After 2-4 weeks in the light, callus begin to organize, and form shoots. Shoots are removed from surrounding callus/media and gently transferred to RM3 media (½×MS salts, Nitsch and Nitsch vitamins, 1% sucrose+50 ppm hygromycin B) in phytatrays (Sigma Chemical Co., St. Louis, Mo.) and incubation is continued using the same conditions as described in the previous step.

Plants are transferred from RM3 to 4″ pots containing Metro mix 350 after 2-3 weeks, when sufficient root and shoot growth have occurred.

### Example 9

### Variants of ZmRR5 and ZmRR6

### A. Variant Nucleotide Sequences of ZmRR5 and ZmRR6 (SEQ ID NOS: 1 and 4) That Do Not Alter the Encoded Amino Acid Sequence

The ZmRR5 and ZmRR6 nucleotide sequences set forth in SEQ ID NOS: 1 and 4 are used to generate variant nucleotide sequences having the nucleotide sequence of the open reading frame with about 70%, 76%, 81%, 86%, 92% and 97% nucleotide sequence identity when compared to the starting unaltered ORF nucleotide sequence of SEQ ID NOS: 1 and 4. These functional variants are generated using a standard codon table. While the nucleotide sequence of the variant is altered, the amino acid sequence encoded by the open reading frame does not change.

### B. Variant Amino Acid Sequences of ZmRR5 and ZmRR6

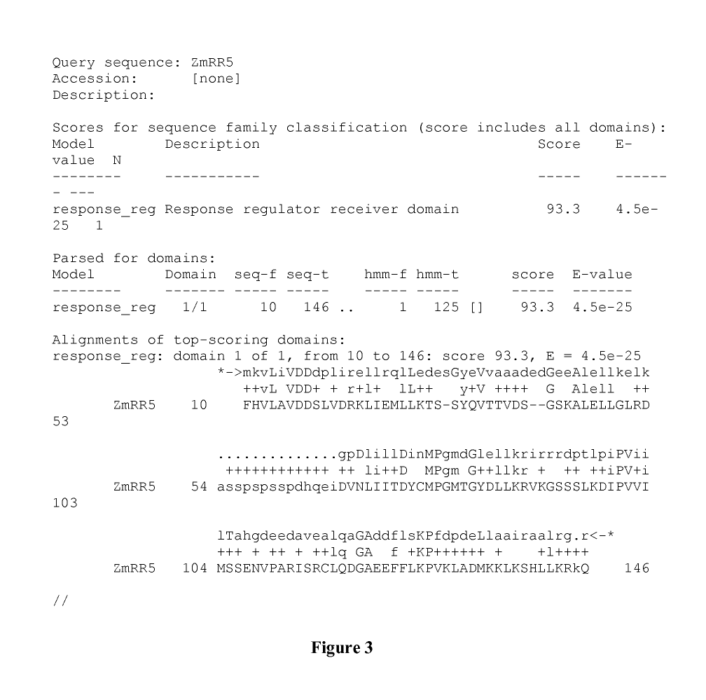

Variant amino acid sequences of ZmRR5 and ZmRR6 are generated. In this example, one amino acid is altered. Specifically, the open reading frame set forth in SEQ ID NO: 1 or 4 is reviewed to determine the appropriate amino acid alteration. The selection of the amino acid to change is made by consulting the protein alignment. See FIG. 2. An amino acid is selected that is deemed not to be under high selection pressure (not highly conserved) and which is rather easily substituted by an amino acid with similar chemical characteristics (i.e., similar functional side-chain). Using the protein alignment set forth in FIG. 2 an appropriate amino acid can be changed. Amino acid residues that show a low percentage of sequence identity among the Zea mays RR proteins are not highlighted. Additional conserved residues can be found in FIGS. 3-6, which provide PFAM and SMART alignments of the type A RR polypeptides. Once the targeted amino acid is identified, the procedure outlined in Example 9A is followed. Variants having about 70%, 75%, 81%, 86%, 92% and 97% amino acid sequence identity to SEQ ID NOS: 2 and 5 are generated using this method.

### C. Additional Variant Amino Acid Sequences of ZmRR5 and ZmRR6

In this example, artificial protein sequences are created having 82%, 87%, 92% and 97% identity relative to the reference protein sequence. This latter effort requires identifying conserved and variable regions from the alignment set forth in FIG. 2 and then the judicious application of an amino acid substitutions table. These parts will be discussed in more detail below.

Largely, the determination of which amino acid sequences are altered is made based on the conserved regions among RR proteins. See, FIGS. 2, 3, 4, 5 and 6. Based on the sequence alignment, the various regions of the ZmRR5 and ZmRR6 that can likely be altered can be determined. It is recognized that conservative substitutions can be made in the conserved regions without altering function. In addition, one of skill will understand that functional variants of the ZmRR5 and ZmRR6 sequences of the invention may also have minor amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 3.

TABLE 3
Substitution Table

| Amino | Strongly Similar and | Rank of Order | |
|---|---|---|---|
| Acid | Optimal Substitution | to Change | Comment |
| I | L, V | 1 | 50:50 substitution |
| L | I, V | 2 | 50:50 substitution |
| V | I, L | 3 | 50:50 substitution |
| A | G | 5 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 15 | |
| Q | N | 15 | |
| F | Y | 16 | |
| M | L | 17 | First methionine cannot |
| | | | change |
| H | | Na | No good substitutes |
| C | | Na | No good substitutes |
| P | | Na | No good substitutes |

First, any conserved amino acid in the protein that should not be changed is identified and “marked off” for insulation from the substitution. The start methionine will of course be added to this list automatically. Next, the changes are made.

H, C, and P are not changed in any circumstance. The changes will occur with isoleucine first, sweeping N-terminal to C-terminal; then leucine, and so on down the list until the desired target is reached. Interim number substitutions can be made so as not to cause reversal of changes. The list is ordered 1-17, so as many isoleucine changes are made as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of ZmRR5 and ZmRR6 are generating having about 82%, 87%, 92% and 97% amino acid identity to the starting unaltered ORF sequence of SEQ ID NOS: 2 and 5.

### Example 10

### Constitutive Expression of ZmRR5 and ZmRR6 Genes in Arabidopsis (SCP1 Promoter)

A proprietary EST sequence, p0128.cpicz20r, which contains a six amino acid duplicated insertion within the ZmRR5 output domain (see, FIG. 7 and SEQ ID NO: 9), was used to transform Arabidopsis thaliana. The construct further comprised the Soybean Constitutive Promoter 1 (SCP1; see, U.S. Pat. No. 6,555,673) and the PINII terminator. In 10 of 12 T0 lines, Arabidopsis plants transformed with the insertional allele showed very slow growth relative to transgenic plants of the same age that had been similarly transformed with the Zm RR6 sequence, when grown under identical conditions.

### Example 11

### Constitutive Expression of ZmRR5 and ZmRR6 genes in Arabidopsis and Zea mays (Ubiquitin Promoter)

The coding sequence for each of the maize response regulators ZM-RR5 and ZM-RR6 (SEQ ID NO: 11 and SEQ ID NO: 4, respectively) was included in constructs PHP23835 (PRO_{ZMUBQ}:ZM-RR5) and PHP23836 (PRO_{ZMUBQ}:ZM-RR6). These constructs were utilized for the constitutive expression of ZmRR5 and ZmRR6 in maize and Arabidopsis transgenics.

Independent transgenic lines of Arabidopsis and maize were analyzed by northern hybridization to identify specific events that had demonstrable transgene expression. For ten separate events of PHP23835 in maize, expression of ZmRR5 was determined by northern blotting using 10 ug of total RNA collected from leaf tissue. The blot was probed with the cDNA for ZmRR5. A range of expression levels was detected in nine of the ten events. For one transgenic event, and for total RNA from leaves of wildtype maize, no expression was detected.

Preliminary examination of T0 maize transgenics for both constructs did not identify gross phenotypic differences, either between transgene positive and negative plants or between transgene positives and other transgenics in the greenhouse at the time.

### Example 12

### Molecular and Phenotypic assays of Arabidopsis transgenics overexpressing ZmRR5 and ZmRR6

Cytokinin signal transduction is mediated by a two-component cascade. This is supported by observations of altered callus growth responses of specific histidine kinase and response regulator mutants (Higuchi, et al., (2004) Proc Natl Acad Sci USA 101:8821-8826; Nishimura, et al., (2004) Plant Cell 16:1365-1377; To, et al., (2004) Plant Cell 16:658-671). A callus growth response assay was developed that could utilize transgenic or mutant tissue that had been transformed by either in planta or ex planta techniques based on a method described by Kakimoto, (1998) J. Plant Research 111:261-265. Transgenics from in planta transformation were evaluated, as this method allowed for characterization of specific transgenic lines and was not influenced by the transformation efficiency of individual hypocotyls.

Response regulator transgenics were tested in callus growth conditions and visually assayed for differences in root and shoot formation. Duplicate, independent hypocotyls of transgenic plants (PRO_{ZMUBQ}:ZM-RR5 and PRO_{ZMUBQ}:ZM-RR6) were grown on callus-inducing media for seven days and subsequently transferred to shoot-inducing media with a range of cytokinin(BA)-to-auxin ratios. A gradient of phenotypic effects on shoot formation was observed; both ZM-RR5 and ZM-RR6 showed a cytokinin hyposensitive phenotype in callus growth assays. The repressive effect of ZM-RR5 and ZM-RR6 on cytokinin-induced callus growth was measured against the normal and enhancing effects observed, respectively, in the wildtype and in tissue transformed with the SCP1 promoter driving ZmRR10 (see, SEQ ID NO: 12).

### Example 13

### Effect of the QA trimer insertion in ZmRR5

All proprietary clones of the ZM-RR5 coding sequence have a six amino acid insertion, a QA trimer, relative to other Zea mays response regulators (see, FIG. 7). To examine a possible functional role of this insertion and that of the putative site of phosphorylation, site-directed deletion (ZM-RR5(VAR1), equivalent to SEQ ID NO: 1) and mutation ZM-RR5(D75N) constructs were created. For the Zm-RR5 (D75N) mutant, the coding sequence was modified to encode asparagine (N) at amino acid position 75, where the conserved aspartate residue normally occurs. See, SEQ ID NO: 13. The modified coding sequence was operably linked to the ubiquitin promoter. Using the Arabidopsis hypocotyl transient transformation protocol, these constructs were evaluated for their ability to influence callus growth in response to exogenous hormones. As expected, the removal of the QA trimer did not influence the ability of ZM-RR5 to inhibit cytokinin-responsive callus growth. This indicates that the insertion of the QA trimer likely has no influence upon cytokinin-responsive callus growth. Further, mutating the putative phosphorylation site also did not influence the ability of ZM-RR5 to inhibit callus growth. This latter observation is in contrast to published results for an Arabidopsis response regulator AT-ARR22 (Kiba, et al., (2004) Plant Cell Physiol. 45:1063-1077).

### Example 14

### Molecular and Phenotypic assays of Zea mays Transgenics Overexpressing ZmRR5 and ZmRR6

Maize transgenics containing constructs for the constitutive expression of ZmRR5 and ZmRR6 genes (PHP23835 and PHP23836) lacked obvious morphological or growth differences relative to other transgenic plants in the greenhouse at the T0 stage.

To determine if the transgenes could influence cytokinin responsiveness, leaf discs from two PHP23835 transgenic lines and one transgenic control were incubated in cytokinin (10 μM BA) for increasing amounts of time (0, 1, 2, 6, or 24 hours) and RNA was prepared, along with 18S RNA. RT-PCR (34 or 37 cycles) was carried out using primers specific to ZmRR7 (see, SEQ ID NO: 14). In these assays, cytokinin-responsive gene expression (ZM-RR7) was hypo-induced in PHP23835 transgenics.

These findings are consistent with the hypothesized antagonistic roles of ZmRR5 and ZmRR7 in cytokinin signal transduction and observations in Arabidopsis callus growth experiments.

Similar analysis of PHP23836 transgenics can also be conducted. Eight selected events (# 1, 2, 4, 5, 6, 8, 11 and 14) of the PHP23835 construct and nine (# 1, 4, 6, 11, 15, 18, 21, 22, 23) of the PHP23836 construct were analyzed using an Agilent 8-pack chip (Agilent Technologies, Palo Alto, Calif., USA) containing 1624 sequences selected from a group of cytokinin-related, ABA-related and drought-related maize genes. Fifty ng of labeled cDNA was hybridized per dye. Normalization was done using a subset of 100 genes on the arrays that had been predetermined for this purpose.

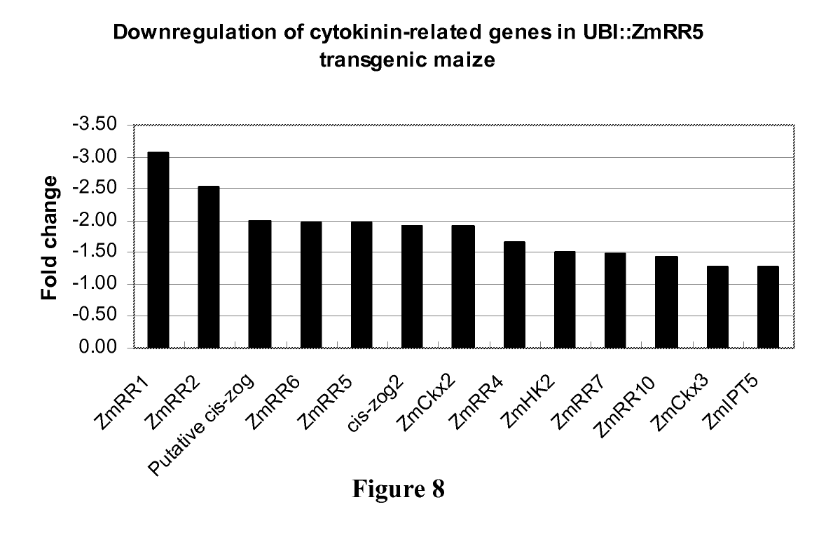

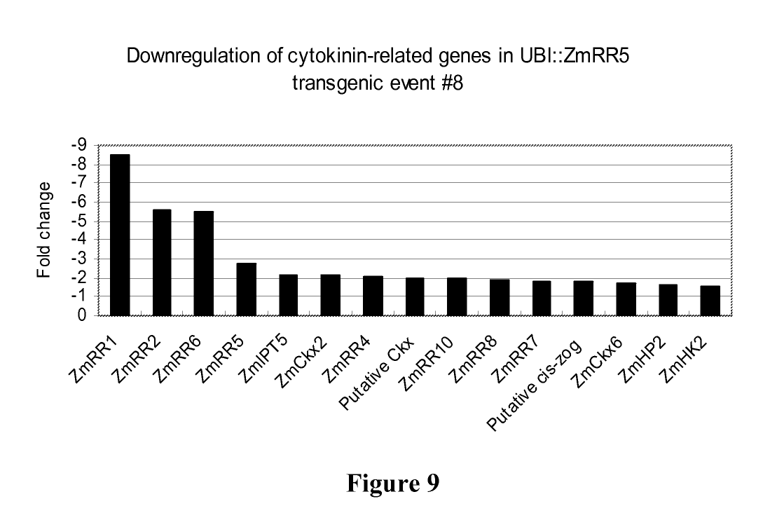

Results for the PHP23835 construct show a consistent down-regulation of several other response regulators and cytokinin-related genes in leaf tissue. FIG. 8 shows the fold-change of a weighted average of down-regulated expression of cytokinin-related genes in transgene positives, relative to a bulk negative of the same transgene construct. ZmRR1 exhibited the greatest fold change in down-regulation of all the 1624 sequences on the chip. FIG. 9 shows the fold change of down-regulated expression of cytokinin-related genes in leaf tissue of transgenic event number 8 of this construct, as compared to the bulk negative. Consistent with the northern blot results of Example 11, events 8, 9 and 11 showed higher transgene expression compared to the other events.

The results of the molecular and phenotypic assays indicate that ZmRR5 is a negative regulator or repressor of cytokinin-response. Cytokinin-mediated growth responses normally observed for wild type Arabidopsis callus are prevented in transgenic Arabidopsis calli containing the PHP23835 construct that overexpresses ZmRR5, as described in Example 13. The same observation is true for ZmRR6 as well. However, while the effect of ZmRR5 overexpression in maize has a distinct effect in reducing cytokinin-related gene expression, as shown in FIGS. 8 and 9, this effect is not as pronounced in the case of ZmRR6.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for affecting growth of a plant, comprising introducing into said plant an expression construct directing modulation of expression of a polynucleotide, said polynucleotide comprising a nucleotide sequence selected from the group consisting of:
(a) a nucleotide sequence comprising SEQ ID NO: 1, 4 or 11;
(b) a nucleotide sequence encoding an amino acid sequence comprising SEQ ID NO: 2, 5 or 9;
(c) a nucleotide sequence having at least 85% sequence identity to SEQ ID NO: 1, 4 or 11, wherein said polynucleotide encodes a polypeptide having response regulator activity; and
(d) a nucleotide sequence that hybridizes under stringent conditions to the complement of the polynucleotide of a), wherein said stringent conditions comprise hybridization in 50% formamide, 1 M NaCl, 1% SDS at 37° C., and a wash in 0.1×SSC at 60° C. to 65° C.;
wherein said plant exhibits an altered phenotype.

2. The method of claim 1, wherein said expression construct further comprises a tissue-specific promoter, a constitutive promoter, or an inducible promoter.

3. The method of claim 2, wherein said tissue-preferred promoter is an immature ear-preferred promoter, a kernel-preferred promoter, a seed-preferred promoter, a shoot-preferred promoter, a leaf-preferred promoter, or a root-preferred promoter.

4. The method of claim 1, wherein said modulation results in decreased expression of the polynucleotide.

5. The method of claim 4, wherein said modulation is accomplished by a technique selected from the group consisting of sense suppression, antisense suppression, double-stranded RNA interference, hairpin RNA interference, and miRNA interference.

6. The method of claim 1, wherein said modulation results in a phenotypic change in the plant with respect to:
a) stress tolerance;
b) seed set during abiotic stress;
c) plant yield;
d) plant vigor;
e) shoot growth;
f) leaf senescence;
g) shoot regeneration; or
h) root growth.

7. The method of claim 1, wherein said modulation comprises a reduction or elimination of a polypeptide encoded by said polynucleotide.

8. The method of claim 1, wherein modulation comprises an increase in the level of a polypeptide encoded by said polynucleotide.

9. The method of claim 1, wherein said plant is a dicot.

10. The method of claim 1, wherein said plant is a monocot.

11. The method of claim 10, wherein said monocot is maize, wheat, rice, barley, sorghum, or rye.

12. A method for enhancing the amount or rate of in vitro callus growth comprising transforming said callus, or a progenitor cell, with an expression construct directing modulation of expression of a polynucleotide, said polynucleotide comprising a nucleotide sequence selected from the group consisting of:
(a) a nucleotide sequence comprising SEQ ID NO: 1, 4 or 11;
(b) a nucleotide sequence encoding an amino acid sequence comprising SEQ ID NO: 2, 5 or 9;
(c) a nucleotide sequence having at least 85% sequence identity to SEQ ID NO: 1, 4 or 11, wherein said polynucleotide encodes a polypeptide having response regulator activity; and
(d) a nucleotide sequence that hybridizes under stringent conditions to the complement of the polynucleotide of a), wherein said stringent conditions comprise hybridization in 50% formamide, 1 M NaCl, 1% SDS at 37° C., and a wash in 0.1×SSC at 60° C. to 65° C.;
wherein the amount or rate of callus growth is increased.

13. The method of claim 12, wherein said modulation results in decreased expression of the polynucleotide.

14. The method of claim 12, further comprising callus growth on media with reduced levels of cytokinin.


SEQUENCE LISTING (23 sequences)

SEQ ID NO: 1 - DNA, 711 bp, Zea mays
  CDS (1)...(711): ZmRR5
atgacggtgccggatgccgagtcgcgcttccatgtcctcgcggtggacgacagcctcgtcgacaggaaactcatcgagatgctgctcaagacctcgtcctaccaagtgaccacggtggattccgggagcaaggcgctggagctgctggggctgagggacgcgtcgtcgccgtctccgtcctcgcctgaccaccaggagatcgacgtgaatctcatcatcactgactactgcatgccaggcatgacaggatacgatctgctcaagagagtgaaggggtcctcctcgctcaaggacattcctgtggtgatcatgtcgtctgagaatgtgcctgcccggatcagcaggtgcttgcaagacggcgcggaggagttcttcctgaagcccgtgaagctggccgacatgaagaagctcaagtcgcacctgctgaaacggaagcagcccaaggaggcgcaggcgcagcagggacaggcggtggagctggagcctgagcagcagctggacccgcgcgcgcagccggcgcacgacgcggaggaaaccgcggcagagccgccgccggccgcatccaacggaaccgccgacggcggcaacaagaggaaggcggcggccatggaggaggaggggatgctggccgtgatgacggtggcggcgccggagagcagcaccaagccgaggctgtccaccaccagcaacagcctggcggtggagacctga

SEQ ID NO: 2 - protein, 236 aa, Zea mays
MTVPDAESRFHVLAVDDSLVDRKLIEMLLKTSSYQVTTVDSGSKALELLGLRDASSPSPSSPDHQEIDVNLIITDYCMPGMTGYDLLKRVKGSSSLKDIPVVIMSSENVPARISRCLQDGAEEFFLKPVKLADMKKLKSHLLKRKQPKEAQAQQGQAVELEPEQQLDPRAQPAHDAEETAAEPPPAASNGTADGGNKRKAAAMEEEGMLAVMTVAAPESSTKPRLSTTSNSLAVET

SEQ ID NO: 3 - DNA, 1654 bp, Zea mays
  misc_feature (0)...(0): full length ZmRR5 (GenBank AB042267)
gcaaagccacgaaccaactcatcgggcgcggagaccaccgcggggggaaaaagcgagggaggggggagaggactgaggagagaaatcatggagccgatttgctgtggagattcgggcgcgtgatccggtcggatcttgctttcccggggggcgatcgaggccacgccgctgctgccgcggtgccctccctctgggctctggcctctgcgtgtgacgtgccgtgccatggcgctcgccctcgcctatatcccccgcctccctcgccccgccgctggccgccgcattcagtaaaaagccacctaccctaccctcctgcgctggtactacttactacctagccaggccaaggagcccaacgaggggaaccgctgcggtaggcgcgcgctgctcgtcccacccacgccgccatgacggtgccggatgccgagtcgcgcttccatgtcctcgcggtggacgacagcctcgtcgacaggaaactcatcgagatgctgctcaagacctcgtcctaccaagtgaccacggtggattccgggagcaaggcgctggagctgctggggctgagggacgcgtcgtcgccgtctccgtcctcgcctgaccaccaggagatcgacgtgaatctcatcatcactgactactgcatgccaggcatgacaggatacgatctgctcaagagagtgaaggggtcctcctcgctcaaggacattcctgtggtgatcatgtcgtctgagaatgtgcctgcccggatcagcaggtgcttgcaagacggcgcggaggagttcttcctgaagcccgtgaagctggccgacatgaagaagctcaagtcgcacctgctgaaacggaagcagcccaaggaggcgcaggcgcagcagggacaggcggtggagctggagcctgagcagcagctggacccgcgcgcgcagccggcgcacgacgcggaggaaaccgcggcagagccgccgccggccgcatccaacggaaccgccgacggcggcaacaagaggaaggcggcggccatggaggaggaggggatgctggccgtgatgacggtggcggcgccggagagcagcaccaagccgaggctgtccaccaccagcaacagcctggcggtggagacctgagctgagaatcggacggcgaccggatcacatactactacctacgtacattaatccaccattgcgggcagtcgagaaccaaccgaccaaacccaggccaccgccgagaaagagctcgtcctccttttctttcgctggttcttttggttctcggagctaattaatggaatgaaggtgtgagctcctggggatggaggggcggttaccggctctaagtgaagaacaattacgacgtaccttacctagcggctgctgcttcggccggcaaattttgttgtgctcgtggtcatggacacccttgtgttcgttaggagggggtcgggtggaacggtgaaaaggaagctgtagatagaaaaggcaggcgtctcatgctcttattgtctagtctaatttttaattttactcggcttattaatcacggtactaactggctgcctcaaaacctgtactggatcagttggctgctgatgttgttttcgctcagttcttttcccacaaatatgtacactcaatattcaaataaactcttttccaatatc

SEQ ID NO: 4 - DNA, 708 bp, Zea mays
  CDS (1)...(708): ZmRR6
atgccaagccaaagcacacacacctcctccagcctttcttcctctacagccccaattcttccctgcagctctgctgctgccttgtttcgatcggtcatggcggcggtggccacagagactccgttccatgtcctggcggtggacgacagcctcccggacaggaagctcatcgagaggctcctcaagacctcttccttccaagtgaccactgtcgactccgggagcaaggcgctgcagttcctgggcctccatgaccaggacagcacggttcctcctgtccacacgcaccagctggatgtggctgccaatcaggatgtggctgtgaacctgatcatcacagactactgcatgcctggcatgacaggatatgacctgctcaagaagatcaaggagtcgtcgtctctcagagatatcccggtggtgatcatgtcctctgagaacattccttcaaggatcaataggtgcctggaggaaggagctgacgagttcttcctaaaacctgtgcggctatcagacatgaacaagctcaagccccacatactgaaaagcagatgcaaccaggaacagcaccagcaaagtgacagtcacagtggcgaacgcaggaaccccacaatcagcagcagcgatagcataaacaaccgcaagagaaagggtgcaggcaccgaagaaatcttgccccagctggcaaacagatcaaggcacagttaa

SEQ ID NO: 5 - protein, 235 aa, Zea mays
MPSQSTHTSSSLSSSTAPILPCSSAAALFRSVMAAVATETPFHVLAVDDSLPDRKLIERLLKTSSFQVTTVDSGSKALQFLGLHDQDSTVPPVHTHQLDVAANQDVAVNLIITDYCMPGMTGYDLLKKIKESSSLRDIPVVIMSSENIPSRINRCLEEGADEFFLKPVRLSDMNKLKPHILKSRCNQEQHQQSDSHSGERRNPTISSSDSINNRKRKGAGTEEILPQLANRSRHS

SEQ ID NO: 6 - DNA, 1158 bp, Zea mays
  misc_feature (0)...(0): full length ZmRR6 (GenBank AB042268)
cagaaagcgaagcacaagcctctgatgccaagccaaagcacacacacctcctccagcctttcttcctctacagccccaattcttccctgcagctctgctgctgccttgtttcgatcggtcatggcggcggtggccacagagactccgttccatgtcctggcggtggacgacagcctcccggacaggaagctcatcgagaggctcctcaagacctcttccttccaagtgaccactgtcgactccgggagcaaggcgctgcagttcctgggcctccatgaccaggacagcacggttcctcctgtccacacgcaccagctggatgtggctgccaatcaggatgtggctgtgaacctgatcatcacagactactgcatgcctggcatgacaggatatgacctgctcaagaagatcaaggagtcgtcgtctctcagagatatcccggtggtgatcatgtcctctgagaacattccttcaaggatcaataggtgcctggaggaaggagctgacgagttcttcctaaaacctgtgcggctatcagacatgaacaagctcaagccccacatactgaaaagcagatgcaaccaggaacagcaccagcaaagtgacagtcacagtggcgaacgcaggaaccccacaatcagcagcagcgatagcataaacaaccgcaagagaaagggtgcaggcaccgaagaaatcttgccccagctggcaaacagatcaaggcacagttaactgagaactgactagtacagctaaaaccttttctttttcactttactttggcctactgatttgtaacgtagatgttagccctggatttgcaaaacggaacggaatctgtaaactgatactgcttgagtcgaatcgatcgaagtgattctgcaccagatctccctatttcatttatcagaatcgaaagccagctgacagagtcacagtaacagcccgttggacatagcagcaggtgccgaagccaagaacatctgaaactgaccgtcggataacctacgatgggagtagccttttactgcttctaagctttagcctggatacaagttgtcactacagtagtcagtagggcagtaacttggtgcatctccaagagttttcaaaacacactgtcaatctagaaatttttggtgatgtagcttctcccac

SEQ ID NO: 7 - protein, 125 aa, Artificial Sequence
  PFAM consensus sequence for Response Regulator domain (PF00072)
MKVLIVDDDPLIRELLRQLLEDESGYEVVAAADEDGEEALELLKELKGPDLILLDINMPGMDGLELLKRIRRRDPTLPIPVIILTAHGDEEDAVEALQAGADDFLSKPFDPDELLAAIRAALRGR

SEQ ID NO: 8 - protein, 150 aa, Artificial Sequence
  SMART consensus sequence for Response Regulator domain (SM0048)
MRILIVDDDPAAAPLIRELLRNYTVIRLLLEKEGYENKLEIVSVVDEADGETLIKEIAEALELLQEEKDKGGDATTAASEKPDLILLDIMMPGDKLELGGMDGLELLRRIRPIIFLTARGDEEDRVRALEAGADDYLTKPFSPEELLARI

SEQ ID NO: 9 - protein, 242 aa, Zea mays
  VARIANT (1)...(242): ZmRR5 insertional allele (positions 150-155)
MTVPDAESRFHVLAVDDSLVDRKLIEMLLKTSSYQVTTVDSGSKALELLGLRDASSPSPSSPDHQEIDVNLIITDYCMPGMTGYDLLKRMKGSSSLKDIPVVIMSSENVPARISRCLQDGAEEFFLKPVKLADMKKLKSHLLKRKQPKEAQAQAQAQAQQGQAVELEPEQQLDPRTQPAHDAEETAAEPPPAASNGTTDGGNKRKAAAMEEEGMLAVMTVAAPESSTKPRLSTTTSSLAVET

SEQ ID NO: 10 - protein, 232 aa, Artificial Sequence
  consensus sequence from Figure 7
MYVPDAESRFHVLAVDDSLVDRKLIEMLLKTSSYQVTTVDSGSKALELLGLRDASSPSPSSPDHQEIDVNLIITDYCMPGMTGYDLLKRMKGSSSLKDIPVVIMSSENVPARISRCLQDGAEEFFLKPVKLADMKKLKSHLLKRKQPKAQAQQGQAVELEPEQQLDPRQPAHDAEETAAEPPPAASNGTDGGNKRKAAAMEEEGMLAVMTVAAPESSTKPRLSTTSSLAVET

SEQ ID NO: 11 - DNA, 726 bp, Zea mays
  CDS (1)...(726): cds for ZmRR5 insertional allele
atgacggtgccagatgccgagtcgcgcttccatgtcctcgcggtggacgacagcctcgtcgacaggaaactcatcgagatgctgctcaagacctcgtcctaccaagtgaccacggtggattccgggagcaaggcgctggagctgctggggctgagggacgcgtcgtcgccgtctccgtcctcgcctgaccaccaggagatcgacgtgaatctcatcatcactgactactgcatgccaggcatgacaggatacgatctgctcaagagaatgaaggggtcctcttcgctcaaggacattcctgtggtgatcatgtcgtctgagaatgtgcctgcccggatcagcaggtgcttgcaagacggcgcggaggagttcttcctgaagcccgtgaagctggccgacatgaagaagctcaagtcgcacctgctgaaacggaagcagcccaaggaggcgcaggcgcaggcgcaggcgcaggcgcagcagggacaggcggtggagctggagcctgagcagcagctggacccgcgcacgcagccggcgcacgacgcggaggaaaccgcggcagagccgccgccggccgcatccaacggaaccaccgatggcggcaacaagaggaaggcggcagccatggaggaggaggggatgctggccgtgatgacggtggcggcgccggagagcagcaccaagccgaggctgtccaccaccaccagcagcctggcggtggaaacc

SEQ ID NO: 12 - DNA, 2061 bp, Zea mays
  CDS (1)...(2061): ZmRR10 (GenBank AB071695)
atggcggcggcagaggcgcggggaggggagttccccgtgggcatgaaggtgctggttgtggacgacgacccgacgtgcctcgttgtgctcaagaggatgctccttgagtgccgatatgacgtgacaacatgtcctcaggctacaagagcactaactatgttgcgagagaataggcgtggttttgatgttataataagtgatgtccacatgccggatatggacggattcaggctacttgaacttgtaggccttgagatggaccttccagttatcatgatgtctgctgattcaagaacggatattgtaatgaacggagttaagcatggagcatgtgactatttaataaaacctgtcagaatggaggagctgaaaaacatctggcaacatgttattaggaaaaaatttaatgaaaacaaggaccatgagcattctggtagcctagatgacaccgatcgtaacagaccaaccaataatgataatgaatacgcttcctccgcgaatgatggaggtgatggcagctggaaatctcagagaaagaaaagagagaaagaagatgatgaaactgacctcgaaaatggtgatccttcttctacatcaaagaaaccaagagttgtttggtcagttgagcttcatcaacaatttgtgaatgcagtcaatcacctcgggatagacaaagctgtcccaaagaaaattttggaattgatgaatgtccctggcttaaccagggaaaatgttgccagccatttgcagaaattcagactctacctgaagagaattgctcagcatcatgcaggaatacctcatccatttgttgcgcctgtatctagtgctaacgttgctccgttaggaggactggaattccaagctttggctgcttctggtcagatccctcctcaagctctggctgctttgcaggatgaactccttggtcgacctacaagcagtttggcgttgcctggaagggaccagtcatctttgcgagtggctgcaaccaaaggaaacaagcaccatgaagaaagagaaatagcatttggtcaacccatatacaagtgtcagaataatgcatatggtgcattccctcaaagcagcccagcagttggaggattgcaaccttttgcagcttggcccaataacaaagttggtatgcctgattcaacaagcacattgggaaatgtgggcaattctcaaaatagcaatatgctattgcatgaattgcagcaacagccagacaccttgctgttaggaacccttcacaatattgatgccaaaccttctggtgtagttatgtcatctcagtcgttaaatacattcccggctagtgagggtatctcacctaatcaaaatcccttgattataccatctcaacccccaagttttgtgtcatcaattcctccatccatgaaacatgaatctcttcttggattaccttcaacgtcaaccagtctgttgggcgggcttgatatggttaatcaagcttcaacaagtcaggctttgattagtagccatggaacaaatcttcctggtctcatgaaccgtagctcaaatgcaatcccttcaccaggaattagtaattttcaaagtggaaatattcattatgttgttaatcagaacgctatgggagttagctctaggccaccaggtgttctaaagaccgagagcactgactcactgagttgtagttatggctatattggtggtagcaccagtgtggactctggcttgttctcttctcagtccaaaaatccacagtatggtctactgcagaatcaaaatgatgttaacggcagctggtcgccttcacaagattttgatagttttggaaattctcttgggcaaggccaccctggtaccacttcatctaacttccagagttccgcccttgggaagttgcctgaccaaggacgagggagaaatcatgggtttgtcgggaaaggcacttgcattccaagccgctttgcagtggatgaggttgaatctccaactaataacttgagccacagcattggaaacagtggagacatagtgaaccccgacatatttggatttagtggacatatgtga

SEQ ID NO: 13 - protein, 236 aa, Zea mays
  VARIANT (1)...(236): D75N mutant of ZmRR5
MTVPDAESRFHVLAVDDSLVDRKLIEMLLKTSSYQVTTVDSGSKALELLGLRDASSPSPSSPDHQEIDVNLIITNYCMPGMTGYDLLKRVKGSSSLKDIPVVIMSSENVPARISRCLQDGAEEFFLKPVKLADMKKLKSHLLKRKQPKEAQAQQGQAVELEPEQQLDPRAQPAHDAEETAAEPPPAASNGTADGGNKRKAAAMEEEGMLAVMTVAAPESSTKPRLSTTSNSLAVET

SEQ ID NO: 14 - DNA, 1058 bp, Zea mays
  misc_feature (1)...(1058): full-length ZmRR7 (GenBank AB042269)
ccgctctctctctgcaggcctgcactgtgaggagactgtgacccggggacggagagctcctttcttctctctcatcgctccaccccttatttaccggtcggttgttcctgggccgtcccgtcccgtcgtctcgcctgggttctcgcttgcttctcaccatcactaccaccgtccacaccatcgcgagggcctggagggaagaggatgggacagggaggagaagtgaaggcggcgccggccgtgagggtgctggtggtggacgactcccccgtcgaccgcaaggtcgtggagctgctgctccggaaccacaaccaccagggcggcgccgcgccattccacgttaccgccgtcgacagcggcaagaaggcaatggagcatctccggctgatggagcaaggcggccagctggattcatgtgccgctgatgctaatcggataaccattgacatcgtgctcacggactactgcatgccggagatgactggctacgacctgctcaaggccatcaaggcgctgagctcccctaacccgatcccggtggtggtgatgtcgtcggagaacgagccccagaggatcagcagatgcctaaccgccggtgccgaggatttcatcctcaagccactcaagaccaaggacgtgcagcgcctgcgcaactgctccagcgccgctagaccaagggacgacgcggcccaccagtgcgagagcttgaggagcagcagcagaaagctgcggtcggatcaggtcgccgcgcctgcccacagatcgcaaccgacgacgggattaaccatggtgctgcatgcctccagcattgagctctcgcactacttccagttcctcctcaagttcgtgctgctcgcgtacgcggtgctgtgcctgaccgagctcctccacagatggtccaatggctccgtcctctccctgtggtctgcatgaggtcgactggaaccgccatgcggtattattagtgtaaccactaaggctccaggctttcagttcaattccccccctgtaatccagtcctggcatgaggagccaccagttcttgctcgccgctccct

SEQ ID NO: 15 - protein, 157 aa, Zea mays
MAAAAPAPASVAPSSAPKATGDSRKTVVSVDASELEKHVLAVDDSSVDRAVIARILRGSRYRVTAVESATRALELLALGLLPDVSMIITDYWMPGMTGYELLKCVKESAALRGIPVVIMSSENVPTRITRCLEEGAEGFLLKPVRPADVSRLCSRIR

SEQ ID NO: 16 - protein, 157 aa, Zea mays
MAAAATATPSVAPESGDRKAVAPPVDAVDLELELEEKHVLAVDDSSVDRAVIAKILRSSKYRVTTVDSATRALELLALGLVPDVNMIITDYWMPGMTGYELLKHVKESSALRAIPVVIMSSENVPTRISRCLEEGAEDFLLKPVRPADVSRLCSRIR

SEQ ID NO: 17 - protein, 135 aa, Zea mays
MASRKCLGGEGSAPAPHVLAVDDSSVDRAVIAGILRSSQFRVTAVDSGKRALELLGTEPNVSMIITDYWMPEMTGYELLKKIKESSRLKEIPVVIMSSENVPTRINRCLEEGAEDFLLKPVRPSDVSRLCSRVLR

SEQ ID NO: 18 - protein, 235 aa, Zea mays
MTVLDAESRFHVLAVDDSIIDRKLIEMLLKSSSYQVTTVESGNKALELLGLRDNGAEDASPPSSSSSSSSSSSPDHQEIDVSLIITDYCMPGMTGYDLLKRVKGSSSLKDIPVVIMSSENVPARISRCLQDGAEEFFLKPVKPADMKKLKSHLLKRKQPKQKNKPQAQAVESPEQQLDPHPQPVHELELEEAAADRNGVNKRKAAAMEEGLTVVVTAPESTKPRRLSTSSLTVET

SEQ ID NO: 19 - protein, 236 aa, Zea mays; identical to SEQ ID NO: 2

SEQ ID NO: 20 - protein, 235 aa, Zea mays; identical to SEQ ID NO: 5

SEQ ID NO: 21 - protein, 242 aa, Zea mays
MGQGGEVKAAPAVRVLVVDDSPVDRKVVELLLRNHNHQGGAAPFHVTAVDSGKKAMEHLRLMEQGGQLDSCAADANRITIDIVLTDYCMPEMTGYDLLKAIKALSSPNPIPVVVMSSENEPQRISRCLTAGAEDFILKPLKTKDVQRLRNCSSAARPRDDAAHQCESLRSSSRKLRSDQVAAPAHRSQPTTGLTMVLHASSIELSHYFQFLLKFVLLAYAVLCLTELLHRWSNGSVLSLWSA

SEQ ID NO: 22 - protein, 120 aa, Artificial Sequence
  Consensus sequence of Figure 2
MVAEHVLAVDDSVDRKVIELLRSSYVTTVDSGSKALELLGLDPQIDVLIITDYCMPGMTGYDLLKKVKESSSLKDIPVVIMSSENVPTRISRCLEEGAEDFLIKPVRPADVRLKSILKKD

SEQ ID NO: 23 - protein, 232 aa, Artificial Sequence
  Consensus sequence shown in Figure 7
MTVPDAESRFHVLAVDDSLVDRKLIEMLLKTSSYQVTTVDSGSKALELLGLRDASSPSPSSPDHQEIDVNLIITDYCMPGMTGYDLLKRMKGSSSLKDIPVVIMSSENVPARISRCLQDGAEEFFLKPVKLADMKKLKSHLLKRKQPKAQAQQGQAVELEPEQQLDPRQPAHDAEETAAEPPPAASNGTDGGNKRKAAAMEEEGMLAVMTVAAPESSTKPRLSTTSSLAVET